SEQUENCE LISTING (4 sequences)

SEQ ID NO: 1 - protein, 12 aa, Artificial Sequence
  Synthetic Peptide
KFFKKFFKFFKK

SEQ ID NO: 2 - protein, 11 aa, Artificial Sequence
  Synthetic Peptide
WKKWWKKWWKK

SEQ ID NO: 3 - protein, 11 aa, Artificial Sequence
  Synthetic Peptide
KKKKFFFKFFK

SEQ ID NO: 4 - protein, 12 aa, Artificial Sequence
  Synthetic Peptide
WWKKWWKKWWKK

(19) United States
(12) Patent Application Publication
(10) Pub. No.: US 2016/0083429 A1
(43) Pub. Date: Mar. 24, 2016

(54) COMPOSITIONS AND METHODS FOR ENDOTOXIN NEUTRALIZATION

(71) Applicant: Immunotrex Biologics, Inc., North Andover, MA (US)

(72) Inventor: Syed K. Hasan, Boston, MA (US)

(21) Appl. No.: 14/863,943

(22) Filed: Sep. 24, 2015

Related U.S. Application Data
(60) Provisional application No. 62/054,792, filed on Sep. 24, 2014.

(51) Int. Cl. C07K 7/08; C07K 7/06; A61K 38/10; A61K 45/06; A61K 38/08

(52) U.S. Cl. CPC C07K 7/08; A61K 45/06; A61K 38/08; A61K 38/10; C07K 7/06

(57) ABSTRACT

Peptide therapeutic agents are provided that bind to lipid A and neutralize the injurious effects of endotoxin.

### RELATED APPLICATIONS

This application claims the benefit of U.S. provisional application No. 62/054,792, filed Sep. 24, 2014, which is incorporated herein by reference in its entirety.

### BACKGROUND OF THE INVENTION

1. Field of the Invention

Peptide therapeutic agents are provided that bind to lipid A and neutralize the injurious effects of endotoxin.

2. Description of the Related Art

Severe sepsis is one of the most significant challenges in critical care. Each year, more than 750,000 people in the U.S. will develop severe sepsis, more than AMI, lung cancer, and other commonly known causes of death in the hospital; more than 215,000 will die from the condition. Treating patients with severe sepsis costs U.S. hospitals nearly $17 billion a year (Aird, 2003); with standard supportive care alone, mortality remains unacceptably high, at 28-50%. (Natanson, 1998). There is an unmet need for additional effective treatments for sepsis.

### SUMMARY OF THE INVENTION

Peptide therapeutic agents are provided that bind to lipid A and neutralize the injurious effects of endotoxin.

In some embodiments, a peptide is provided according to Formula I:

X—(O¹)ₘ—(K)ₙ—(O²)_{n′}—(K)ⱼ—(O³)_{j′}—(K)_{j″}—(O⁴)_{m′}—(K)_{m″}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; each optionally substituted with halo; m, m′, and m″ are each independently 0, 1, or 2; n and n′ are each independently 1, 2, 3, or 4; and j, j′, and j″ are each independently 1 or 2.

In some embodiments, the peptide of formula I, or pharmaceutically acceptable salt thereof, is provided comprising O¹, O², O³, and O⁴ that are each independently selected from a Phe or a Trp residue.

In some embodiments, the peptide of formula I, or pharmaceutically acceptable salt thereof, is provided comprising O¹, O², O³, and O⁴ that are each a Tip residue.

In some embodiments, the peptide of formula I, or pharmaceutically acceptable salt thereof, is provided comprising O¹, O², O³, and O⁴ are each a Phe residue.

In some embodiments, the peptide of formula I, or pharmaceutically acceptable salt thereof, is provided comprising X that is H and Z that is OH.

In some embodiments, the peptide of formula I, or pharmaceutically acceptable salt thereof, is provided comprising n′=2 or 3; j=1 or 2; j′=2; and j″=1 or 2.

In some embodiments, the peptide of formula I, or pharmaceutically acceptable salt thereof, is provided comprising X═H; O¹, O², and O³ are each a Tip residue; O⁴ is absent; Z is OH; m=1; n, n′, j, j′, j″ are each=2; and m′ and m″ both=0.

In some embodiments, the peptide of formula I, or pharmaceutically acceptable salt thereof, is provided comprising X═H; O¹ is absent; O², O³ and O⁴ are each a Phe residue; Z is OH; m=0; n and j″ each=1; and n′, j, j′, m′, and m″each=2.

In some embodiments, the peptide of formula I, or pharmaceutically acceptable salt thereof, is provided comprising X═H; O¹ and O⁴ are each absent; O² and O³ are each a Phe residue; Z is OH; m=0; n=4, n′=3; j=1; j′=2; j″=1; m′ and m″=0.

In some embodiments, a peptide of formula I, or pharmaceutically acceptable salt thereof, is provided comprising an amino acid sequence selected from the group consisting of SEQ ID NO: 1, 2, 3, and 4.

In some embodiments, a method of inhibiting effects of lipid A on a cell is provided, the method comprising exposing the cell to an effective amount of a peptide according to formula I:

X—(O¹)ₘ—(K)ₙ—(O²)_{n′}—(K)ⱼ—(O³)_{j′}—(K)_{j″}—(O⁴)_{m′}—(K)_{m″}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2. In some aspects, each of O¹, O², O³, and O⁴ is independently selected from a Phe or a Trp residue. In other aspects, each of O¹, O², O³, and O⁴ is a Trp residue. In further aspects, each of O¹, O², O³, and O⁴ is a Phe residue. In some aspects, X is H and Z is OH. In additional aspects, n′=2 or 3; j=1 or 2; j′=2; and j″=1 or 2.

In some embodiments, a method of inhibiting effects of lipid A on a cell is provided, the method comprising exposing the cell to an effective amount of a peptide according to formula I, or a pharmaceutically acceptable salt thereof, wherein X═H; O¹, O², and O³ are each a Trp residue; O⁴ is absent; Z is OH; m=1; n, n′, j, j′, j″ are each=2; and m′ and m″ both=0.

In some embodiments, a method of inhibiting effects of lipid A on a cell is provided, the method comprising exposing the cell to an effective amount of a peptide according to formula I, or a pharmaceutically acceptable salt thereof, wherein X═H; O¹ is absent; O², O³ and O⁴ are each a Phe residue; Z is OH; m=0; n and j″ each=1; and n′, j, j′, m′, and m″ each=2.

In some embodiments, a method of inhibiting effects of lipid A on a cell is provided, the method comprising exposing the cell to an effective amount of a peptide according to formula I, or a pharmaceutically acceptable salt thereof, wherein X═H; O¹ and O⁴ are each absent; O² and O³ are each a Phe residue; Z is OH; m=0; n=4, n′=3; j=1; j′=2; j″=1; m′ and m″=0.

In some embodiments, a method of inhibiting effects of lipid A on a cell is provided, the method comprising exposing the cell to an effective amount of a peptide according to formula I, or a pharmaceutically acceptable salt thereof, wherein the peptide has an amino acid sequence selected from SEQ ID NO: 1, 2, 3, or 4.

In some embodiments, a method of inhibiting effects of lipid A on a cell is provided, the method comprising exposing the cell to an effective amount of a peptide according to formula I, or a pharmaceutically acceptable salt thereof, wherein the peptide has an amino acid sequence of SEQ ID NO: 2.

In some embodiments, a method of inhibiting binding of LPS, endotoxin, or lipid A in a solution to a cell is provided, the method comprising exposing the solution to an effective amount of a peptide of formula I, or pharmaceutically acceptable salt thereof, as provided herein. In some embodiments, a method of inhibiting binding of LPS, endotoxin, or lipid A in a solution to a cell is provided, the method comprising exposing the solution to an effective amount of a peptide of formula I,

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2. In some aspects, each of O¹, O², O³, and O⁴ is independently selected from a Phe or a Trp residue. In other aspects, each of O¹, O², O³, and O⁴ is a Trp residue. In further aspects, each of O¹, O², O³, and O⁴ is a Phe residue. In some aspects, X is H and Z is OH. In additional aspects, n′=2 or 3; j=1 or 2; j′=2; and j″=1 or 2.

In some embodiments, a pharmaceutical composition is provided comprising an effective amount of a peptide according to Formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2; and a pharmaceutically acceptable carrier.

In some embodiments, a pharmaceutical composition is provided comprising an effective amount of a peptide according to Formula I, as provided herein, or a pharmaceutically acceptable salt thereof; and a pharmaceutically acceptable carrier, and further comprising an additional active component selected from one or more of an antibiotic, antioxidant, vasopressor, steroid, or recombinant human activated protein C.

In some embodiments, a pharmaceutical composition is provided comprising an effective amount of a peptide according to Formula I, as provided herein, or a pharmaceutically acceptable salt thereof; and a pharmaceutically acceptable carrier, and further comprising an antibiotic selected from one or more of the group consisting of ceftriaxone, azithromycin, moxifloxacin, piperacillin, tazobactam, tobramycin, ampicillin, Cipro, metronidazole, vancomycin, clindamycin, and ceftazidime.

In some embodiments, a pharmaceutical composition is provided comprising an effective amount of a peptide according to Formula I, as provided herein, or a pharmaceutically acceptable salt thereof; and a pharmaceutically acceptable carrier, and further comprising a vasopressor is selected from one or more of the group consisting of norepinephrine, dopamine, epinephrine, vasopressin, phenylephrine, and dobutamine.

In some embodiments, a pharmaceutical composition is provided comprising an effective amount of a peptide according to Formula I, as provided herein, or a pharmaceutically acceptable salt thereof; and a pharmaceutically acceptable carrier, and further comprising a steroid selected from one or more of the group consisting of dexamethasone, hydrocortisone, and fludrocortisone.

In some embodiments, a method of treating a patient suffering from, or suspected of suffering from, a gram-negative bacterial infection is provided, the method comprising administering to the patient an effective amount of a peptide according to formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2.

In some embodiments, a method is provided for inhibiting binding of LPS, endotoxin, or lipid A in a solution to a cell, the method comprising exposing the solution to an effective amount of a peptide of formula I, or a pharmaceutically acceptable salt thereof, as provided herein.

In some embodiments, a method is provided for absorbing lipopolysaccharide (LPS) present in a solution, comprising contacting the solution with an effective amount of a peptide of formula I, or a pharmaceutically acceptable salt thereof, as provided herein, wherein the effective amount of peptide is one that reduces the amount of LPS, endotoxin or lipid A, in the solution by at least 50%, at least 90%, at least 99%, at least 99.5%, or at least 99.9% compared to the amount of LPS in the original solution. In some embodiments, the amount of LPS in solution is determined by an LAL (Limulus amebocyte lysate) gel-clot, turbidometric or chromogenic test method.

### BRIEF DESCRIPTION OF THE DRAWINGS

FIG. 1A shows a chemical structure of a diphosphoryl Lipid A with Molecular Formula of C₉₄O₂₅N₂P₂H₁₇₈.

FIG. 1B shows a detail of a Kdo2-Lipid A which comprises a phosphorylated glucosamine disaccharide with multiple fatty acid substituents

FIG. 2A shows a molecular model of lipopolysaccharide with antigenic repeats, core and lipid A portions. Lipid A is targeted for binding to the peptides of the invention.

FIG. 2B shows the structures of certain individual amino acids and amino acid sequences for certain peptide embodiments of the invention.

FIG. 3 shows peptide configurations by molecular modeling generated by Mobyle@RPBS. Two views of molecular models for each of three peptides, WK-11, KK-11 and KK-12 are shown. Two of the three peptides, WK-11 and KK-11, exhibit substantial cylindrical configuration.

FIG. 4A shows ESI-MS ionization of peptide structures HK-11, KK-12, and KK-11.

FIG. 4B shows ESI-MS ionization of diphosphoryl Lipid A.

FIG. 5A shows THP-1 cell line in culture after stimulating with LPS; cells darken after exposure to LPS; cells adhere to one another and exhibit a rough membrane.

FIG. 5B shows THP-1 cells stimulated with LPS, and then treated after 24 hrs. with peptide 1 (KK-12)(SEQ ID NO: 1). After treatment, cells do not adhere to one another and cells outer membrane shape and color returns to normal.

FIG. 6 shows electrospray ionization mass spectrometry (ESI-MS) of peptide WK-11 and Lipid-A and detection of complex formation at double circles; the major WK-11-Lipid-A complex exhibits observed MS (ESI) m/z 1173.0865 (M+3).

FIG. 7 shows ESI-MS of peptide KK-12 and Lipid-A and detection of complex formation shown at double circles; the major KK-12-Lipid-A complex exhibits observed MS (ESI) m/z 1157.1163 (M+3).

FIG. 8 shows ESI-MS of peptide KK-11 and Lipid-A and detection of complex formation shown at double circles; the KK-11-Lipid-A complex exhibits observed MS (ESI) m/z 1107.815 (M+3).

FIG. 9 shows ESI-MS ionization of control peptide Bombesin mixed with diphosphoryl Lipid A. No complex formation is observed. Presence of D-lipid-A was confirmed by negative mode ESI-MS.

FIG. 10 shows preliminary ESI-MS competition binding experiment between WK-11 and KK-12 for binding to diphosphoryl-Lipid-A. Two lipid A complexes are observed as shown by double circles. The double circle at right surrounding “1” represents a WK-11-Lipid-A complex observed MS (ESI) m/z 1173.09 (M+3). The double circle surrounding “2” represents KK-12-Lipid-A complex.

FIG. 11 shows ESI-MS ionization signal response total intensity (Iₜₒₜₐₗ) from free WK-11 and KK-12 peptides from CHL/MeOH/H₂O solutions when no lipid D is present. Iₜₒₜₐₗ (WK-11)<Iₜₒₜₐₗ (KK-12) (about 1.11 times higher by total ion count).

FIG. 12 shows ESI-MS ionization following mixing of WK-11 and KK-12 peptides with diphosphoryl lipid A at 0.0032 mg/mL WK-11; 0.0032 mg/mL KK-12; and 0.001 mg/mL D-lipid-A.

FIG. 13 shows ESI-MS ionization following mixing of WK-11 and KK-12 peptides with diphosphoryl lipid A at 0.0032 mg/mL WK-11; 0.0032 mg/mL KK-12; and 0.0025 mg/mL D-lipid-A.

FIG. 14 shows ESI-MS ionization following mixing of WK-11 and KK-12 peptides with diphosphoryl lipid A at 0.0032 mg/mL WK-11; 0.0032 mg/mL KK-12; and 0.0050 mg/mL D-lipid-A.

FIG. 15 shows ESI-MS ionization signal response total intensity (Iₜₒₜₐₗ) from free WK-11 and KK-11 peptides from MeOH/H₂O/0.1% formic acid solution with no D-lipid-A present. Iₜₒₜₐₗ (WK-11)≃Iₜₒₜₐₗ (KK-11).

FIG. 16 shows ESI-MS ionization signal response total intensity (Iₜₒₜₐₗ) from free WK-11 and KK-11 peptides from MeOH/CHL/H₂O solution with no D-lipid-A present. Iₜₒₜₐₗ (WK-11)≃Iₜₒₜₐₗ (KK-11).

FIG. 17 shows ESI-MS ionization following mixing of WK-11 and KK-11 peptides with diphosphoryl lipid A at 0.0032 mg/mL WK-11; 0.0032 mg/mL KK-11; and 0.0025 mg/mL D-lipid-A.

FIG. 18 shows ESI-MS ionization following mixing of WK-11 and KK-11 peptides with diphosphoryl lipid A at 0.0032 mg/mL WK-11; 0.0032 mg/mL KK-11; and 0.005 mg/mL D-lipid-A.

FIG. 19 shows ESI-MS ionization following mixing of KK-12 and KK-11 peptides with diphosphoryl lipid A at 0.0032 mg/mL KK-12; 0.0032 mg/mL KK-11; and 0.005 mg/mL D-lipid-A.

FIG. 20A shows a plot of R values vs. concentration of D-Lipid-A for WK-11 vs. KK-12. Relative binding of WK-11 with D-Lipid-A was greater than relative binding of KK-12 with D-Lipid-A.

FIG. 20B shows a plot of R values vs. concentration of D-Lipid-A for WK-11 vs. KK-11. Relative binding of WK-11 with D-Lipid-A was greater than relative binding of KK-11 with D-Lipid-A.

### DETAILED DESCRIPTION OF THE INVENTION

Peptide therapeutic agents/compounds are provided herein for the treatment of sepsis. These small peptides (typically 11 or 12 amino acids long) neutralize the injurious effects of endotoxin and are thereby capable of blocking the multiple pathophysiologic processes involved in sepsis.

### DEFINITIONS

The following definitions are provided to facilitate understanding of certain terms used frequently herein and are not meant to limit the scope of the present disclosure.

“Amino acid” refers to any of the twenty naturally occurring amino acids. In some embodiments, the amino acid residues may be unmodified or modified in amino acid sequences. Modifications can include natural processes, such as posttranslational processing, or chemical modifications which are generally known in the art. Modifications include but are not limited to: phosphorylation, ubiquitination, acetylation, amidation, glycosylation, covalent attachment of flavin, ADP-ribosylation, cross-linking, iodination, methylation, and other like modifications. In other embodiments, amino acid residue side chains, or N-terminus, or C-terminus, may be modified as otherwise provided herein. In some embodiments, no amino acid residue side chain or N-terminus or C-terminus is otherwise modified, and may exist as protonated, ionized side chains, depending on pH. In some embodiments, the amino acid residues may be present as one or more pharmaceutically acceptable salts.

“Protein,” “peptide,” and “polypeptide” are used interchangeably herein to denote an amino acid polymer joined by peptide amide bonds.

The term “aliphatic” or “aliphatic group” as used herein means a straight-chain or branched C1-12 hydrocarbon chain that is completely saturated or that contains one or more units of unsaturation, or a monocyclic C3-8 hydrocarbon or bicyclic C8-12 hydrocarbon that is completely saturated or that contains one or more units of unsaturation, but which is not aromatic (also referred to herein as “carbocycle” or “cycloalkyl”), that has a single point of attachment to the rest of the molecule wherein any individual ring in said bicyclic ring system has 3-7 members. For example, suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The terms “alkyl,” “alkoxy,” “hydroxyalkyl,” “alkoxyalkyl” and “alkoxycarbonyl,” used alone or as part of a larger moiety include both straight and branched chains containing one to twelve carbon atoms. The terms “alkenyl” and “alkynyl” used alone or as part of a larger moiety shall include both straight and branched chains containing two to twelve carbon atoms.

The terms “haloalkyl,” “haloalkenyl” and “haloalkoxy” means alkyl, alkenyl or alkoxy, as the case may be, substituted with one or more halogen atoms. The term “halogen” or “halo” means F, Cl, Br or I.

The term “heteroatom” means nitrogen, oxygen, or sulfur and includes any oxidized form of nitrogen and sulfur, and the quaternized form of any basic nitrogen.

The term “aryl” used alone or in combination with other terms, refers to monocyclic, bicyclic or tricyclic carbocyclic ring systems having a total of five to fourteen ring members, wherein at least one ring in the system is aromatic and wherein each ring in the system contains 3 to 8 ring members. The term “aryl” may be used interchangeably with the term “aryl ring”. The term “aralkyl” refers to an alkyl group substituted by an aryl. The term “aralkoxy” refers to an alkoxy group substituted by an aryl.

As used herein, where a ring is defined to contain or comprise x to y members, it is understood that the total number of member atoms (e.g., carbon or heteroatoms) making up the ring is x, y or any integer between x and y. By way of example, a ring comprising 3 to 8 carbon or heteroatoms may be a ring containing 3, 4, 5, 6, 7 or 8 ring members.

The term “heterocycloalkyl,” “heterocycle,” “heterocyclyl” or “heterocyclic” as used herein means monocyclic, bicyclic or tricyclic ring systems having 5, 6, 7, 8, 9, 10, 11, 12, 13 or 14 ring members in which one or more ring members is a heteroatom, wherein each ring in the system contains 3, 4, 5, 6, 7 or 8 ring members and is non-aromatic.

The term “heteroaryl,” used alone or in combination with other terms, refers to monocyclic, bicyclic and tricyclic ring systems having a total of 5, 6, 7, 8, 9, 10, 11, 12, 13 or 14 ring members, and wherein: 1) at least one ring in the system is aromatic; 2) at least one ring in the system contains one or more heteroatoms; and 3) each ring in the system contains 3, 4, 5, 6 or 7 ring members. The term “heteroaryl” may be used interchangeably with the term “heteroaryl ring” or the term “heteroaromatic”. Examples of heteroaryl rings include, but are not limited to, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 3-pyrazolyl, 4-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. The term “heteroaralkyl” refers to an alkyl group substituted by a heteroaryl. The term “heteroarylalkoxy” refers to an alkoxy group substituted by a heteroaryl.

An aryl (including aralkyl, aralkoxy, aryloxyalkyl and the like) or heteroaryl (including heteroaralkyl, heteroarylalkoxy and the like) group may contain one or more substituents. Suitable substituents on an unsaturated carbon atom of an aryl, heteroaryl, aralkyl or heteroaralkyl group are selected from halogen; haloalkyl; —CF₃; —R; —OR; —SR; 1,2-methylenedioxy; 1,2-ethylenedioxy; protected OH (such as acyloxy); phenyl (Ph); Ph substituted with R; —O(Ph); —O-(Ph) substituted with R; —CH₂(Ph); —CH₂(Ph) substituted with R; —CH₂CH₂(Ph); —CH₂CH₂(Ph) substituted with R; —NO₂; —CN; —N(R)₂; —NRC(O)R; —NRC(O)N(R)₂; —NRCO₂R; —NRNRC(O)R; —NR—NRC(O)N(R)₂; —NRNRCO₂R;

—C(O)C(O)R; —C(O)CH₂C(O)R; —CO₂R; —C(O)R; —C(O)N(R)₂; —OC(O)N(R)₂; —S(O)₂R; —SO₂N(R)₂; —S(O)R; —NRSO₂N(R)₂; —NRSO₂R; —C(═S)N(R)₂; —C(═NH)—N(R)₂; —(CH₂)_{y}NHC(O)R; —(CH₂)_{y}R; —(CH₂)_{y}NHC(O)NHR; —(CH₂)_{y}NHC(O)OR; —(CH₂)_{y}NHS(O)R; —(CH₂)_{y}NHSO₂R; —(CH₂)_{y}NHC(O)CH((V)_{z}—R)(R); —(CH₂)ₘC(O)NH₂ or —(CH₂)ₘOCH₃ wherein each R is independently selected from hydrogen, optionally substituted aliphatic (preferably C₁₋₆), an unsubstituted heteroaryl or heterocyclic ring (preferably C₅₋₆), phenyl (Ph), —O(Ph), or —CH₂(Ph)-CH₂(Ph), wherein m is 0-4; y is 0-6; z is 0-1; and V is a linker group. When R is aliphatic, it may be substituted with one or more substituents selected from —NH₂, —NH(C₁₋₄ aliphatic), —N(C₁₋₄ aliphatic)₂, —S(O)(C₁₋₄ aliphatic), —SO₂(C₁₋₄ aliphatic), halogen, (C₁₋₄ aliphatic), —OH, —O—(C₁₋₄ aliphatic), —NO₂, —CN, —CO₂H, —CO₂(C₁₋₄ aliphatic), —O(halo C₁₋₄ aliphatic) or -halo(C₁₋₄ aliphatic); wherein each C₁₋₄ aliphatic is unsubstituted.

An aliphatic group or a non-aromatic heterocyclic ring may contain one or more substituents. Suitable substituents on a saturated carbon of an aliphatic group or of a non-aromatic heterocyclic ring are selected from those listed above for the unsaturated carbon of an aryl or heteroaryl group and the following: ═O, ═S, ═NNHR, ═NN(R)₂, ═N—, ═NNHC(O)R, ═NNHCO₂(alkyl), ═NNHSO₂(alkyl), or ═NR, where each R is independently selected from hydrogen or an optionally substituted aliphatic (preferably C₁₋₆). When R is aliphatic, it may be substituted with one or more substituents selected from —NH₂, —NH(C₁₋₄ aliphatic), —N(C₁₋₄ aliphatic)₂, halogen, —OH, —O—(C₁₋₄ aliphatic), —NO₂, —CN, —CO₂H, —CO₂(C₁₋₄ aliphatic), —O(halo C₁₋₄ aliphatic), or -halo(C₁₋₄ aliphatic); wherein each C₁₋₄ aliphatic is unsubstituted.

Substituents on a nitrogen of a non-aromatic heterocyclic ring are selected from

—R, —N(R)₂, —C(O)R, —C(O)OR, —C(O)C(O)R, —C(O)CH₂C(O)R, —SO₂R, —SO₂N(R)₂, —C(═S)N(R)₂, —C(═NH)—N(R)₂ or —NRSO₂R; wherein each R is independently selected from hydrogen, an optionally substituted aliphatic (preferably C₁₋₆), optionally substituted phenyl (Ph), optionally substituted —O(Ph), optionally substituted —CH₂(Ph), optionally substituted —CH₂CH₂(Ph), or an unsubstituted heteroaryl or heterocyclic ring (preferably 5-6 membered). When R is a C₁₋₆ aliphatic group or a phenyl ring, it may be substituted with one or more substituents selected from —NH₂, —NH(C₁₋₄ aliphatic), —N(C₁₋₄ aliphatic)₂, halogen, —(C₁₋₄ aliphatic), —OH, —O—(C₁₋₄ aliphatic), —NO₂, —CN, —CO₂H, —CO₂(C₁₋₄ aliphatic), —O(halo C₁₋₄ aliphatic) or -halo(C₁₋₄ aliphatic); wherein each C₁₋₄ aliphatic is unsubstituted.

The term “treatment” or “treating” refers to any treatment of a pathologic condition in a mammal, particularly a human, and includes: (i) preventing the pathologic condition from occurring in a subject which may be predisposed to the condition but has not yet been diagnosed with the condition and, accordingly, the treatment constitutes prophylactic treatment for the disease condition; (ii) inhibiting the pathologic condition, i.e., arresting its development; (iii) relieving the pathologic condition, i.e., causing regression of the pathologic condition; or (iv) relieving the conditions mediated by the pathologic condition.

The term “therapeutically effective amount” refers to that amount of a compound of the invention that is sufficient to effect treatment, as defined above, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable salts” includes, but is not limited to, salts well known to those skilled in the art, for example, mono-salts (e.g. alkali metal and ammonium salts) and poly salts (e.g. di- or tri-salts,) of the compounds of the invention. Pharmaceutically acceptable salts of compounds of Formula (I) are where, for example, an exchangeable group, such as hydrogen in —OH, —NH—, or —P(═O)(OH)—, is replaced with a pharmaceutically acceptable cation (e.g. a sodium, potassium, or ammonium ion) and can be conveniently be prepared from a corresponding compound of Formula (I) by, for example, reaction with a suitable base. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts may be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example, calcium) salts of carboxylic acids can also be made.

The term “prodrug” or “prodrugs” is used in its ordinary meaning in the art and means a compound of the invention that has its charged moieties masked or protected by another moiety that is designed to be cleaved under particular physiological conditions, leaving the deprotected or unmasked compound of the invention. The use of masking agents is common and well-known in the art and, in particular, masking phosphate or phosphonate groups. All such masking agents are suitable and can be used with the compounds of the invention. Various agents such as acyloxy alkyl esters are described by Srivasta et al., (1984 Bioorganic Chemistry 12, 118-12), and by Freeman et al. (1997 Progress in Medicinal Chemistry 34:112-147) which are each incorporated in their entirety herein by reference; and 3-phthalidyl phosphonate esters are described by Dang Q., et al., (1999 Bioorganic & Med. Chem Letters, 9:1505-1510), which is incorporated in its entirety herein by reference. For example, and not by way of limitation, Srivasta et al. also describe acetoxymethyl, isobutryloxymethyl, and pivaloxymethyl as masking agents. Other suitable masking groups comprising pivaloxyalkyl, e.g., pivaloxymethyl, or a pivaloyloxy group as described by Farquhar D. et al., (1995 J. Med. Chem., 38:488-495) which is incorporated in its entirety herein by reference. Still other masking or protecting agents are described in U.S. Pat. Nos. 4,816,570 and 4,968,788 both of which are incorporated in their entirety herein by reference. Lipid prodrugs are also suitable for use with the compounds of the invention. By non-limiting example, certain lipid prodrugs are described in Hostetler et al., (1997 Biochem. Pharm. 53:1815-1822), and Hostetler et al., 1996 Antiviral Research 31:59-67), both of which are incorporated in their entirety herein by reference. Additional examples of suitable prodrug technology is described in WO 90/00555; WO 96/39831; WO 03/095665A2; U.S. Pat. Nos. 5,411,947; 5,463,092; 6,312,662; 6,716,825; and U.S. Published Patent Application Nos. 2003/0229225 and 2003/0225277 each of which is incorporated in their entirety herein by reference. Such prodrugs may also possess the ability to target the drug compound to a particular tissue within the patient, e.g., liver, as described by Erion et al., (2004 J. Am. Chem. Soc. 126:5154-5163; Erion et al., Am. Soc. Pharm. & Exper. Ther. DOI:10.1124/jept.104.75903 (2004); WO 01/18013 A1; U.S. Pat. No. 6,752,981), each of which is incorporated in their entirety herein by reference. By way of non-limiting example, other prodrugs suitable for use with the compounds of the invention are described in WO 03/090690; U.S. Pat. No. 6,903,081; U.S. Patent Application No. 2005/0171060A1; U.S. Patent Application No. 2002/0004594A1; and by Harris et al., (2002 Antiviral Chem. & Chemo. 12: 293-300; Knaggs et al., 2000 Bioorganic & Med. Chem. Letters 10: 2075-2078) each of which is incorporated in their entirety herein by reference.

Some of the compounds described herein possess one or more chiral (also known as asymmetric) centers, and may lead to optical isomers. All such isomers, as well as diastereomers and enantiomers are included in the present invention. Racemic mixtures of compounds are also included in the present invention. Resolution of such racemic mixtures can be made using standard procedures known in the art. By way of non-limiting example, one of skill in the art can obtain the two enantiomers of the racemic amino acid by using chiral column separation or by proper functionalization followed by enzymatic resolution or by treatment of the racemate with a chiral amine to form a diastereomeric salt and the two diastereomers separated by crystallization. The parent compound can then be liberated from the amine salt by acid treatment. Alternatively, one can obtain the two enantiomers of the racemic final compound by using chiral column separation or by treatment with a chiral amine to form a diastereomeric salt and the two diastereomers separated by crystallization. The parent compound can then be liberated from the amine salt by acid treatment. Another method that can be used to resolve enantiomers of a chiral amino acid is to form a conjugate (e.g. ester) with a chiral moiety (e.g. a chiral alcohol) to produce a mixture of diasteromeric adducts. These adducts can be separated by ordinary (non-chiral) chromatography or by fractional crystallization, then the respective enantiomers of the amino acid liberated by cleavage of the conjugate.

Sepsis

Sepsis is a systemic inflammatory response due to an infection. There may be either clinical suspicion or microbiological evidence of the infection. Severe sepsis is associated with organ dysfunction, hyoperfusion, or hypotension. Hypoperfusion and perfusion abnormalities may include, but are not limited to lactic acidosis, oliguria, or an acute alteration in mental status. Septic shock is severe sepsis induced hypotension despite adequate fluid. Patients receiving inoptropic or vasopressor agents may no longer be hypotensive by the time they manifest hypoperfusion abnormalities or organ dysfunction, but would still be considered to have septic shock. Soong, Sepsis: recognition and treatment. Clinical Medicine 2012, Vol. 12 No. 3: 276-80. The infectious agents in sepsis are usually bacteria, but can also be fungi or viruses. Gram-negative bacteria are the most common infective agents in sepsis. Gram-negative bacteria have an outer membrane containing lipopolysaccharide (LPS), which consists of lipid A, core polysaccharide, and O antigen.

Sepsis may result in a patient from any one or from multiple sources of infection including respiratory, urinary tract, intra-abdominal, biliary, device-related, skin-soft tissue, meningitis, neutropenic fever, or from an unknown source. In many cases, the infection is a gram-negative bacterial infection.

Appropriate cultures are typically taken from the patient prior to administration of antibiotics. Empiric antibiotics and combinations for the initial treatment of sepsis by initial intravenous administration include, but are not limited to, several combinations of antibiotics.

Intravenous administration of antibiotics is typically instituted in the treatment of sepsis prior to obtaining culture results from the patient.

Antibiotics and combinations of antibiotics for use in initial treatment of a patient suffering from sepsis are typically selected from one or more of ceftriazone plus azithromycin, moxifloxacin, piperacillin/tazobactam combination plus tobramycin, azithromycin, ampicillin plus tobramycin, Cipro plus tobramycin, piperacillin plus tobramycin, ampicillin plus metronidazole, vancomycin plus metronidazole, ceftazidime plus metronidazole, vancomycin plus tobramycin, vancomycin plus clindamycin, clindamycin plus tobramycin plus metronidazole, ceftriaxone plus vancomycin, tobramycin plus vancomycin, Cipro plus vancomycin, tobramycin plus vancomycin plus metronidazole, Cipro plus vancomycin plus metronidazole, ceftazidime, ceftazidime plus tobramycin, and vancomycin, or ceftazadime plus metronidazole.

In some embodiments, a steroid drug is also administered intravenously with antibiotics, for example, dexamethasone.

Gram-Negative Bacteria

Medically relevant examples of gram-negative bacilli include a multitude of species. Some of them cause primarily respiratory problems (Hemophilus influenza, Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella enteritidis, Salmonella typhi). Medically relevant gram-negative cocci include the three organisms that cause a sexually transmitted disease (Neisseria gonorrhoeae), a meningitis (Neisseria meningitidis), and respiratory symptoms (Moraxella catarrhalis).

The pathogenic capability of gram-negative bacteria is often associated with certain components of their membrane, in particular, the lipopolysaccharide layer (also known as the LPS or endotoxin layer).

In humans, it is known that the presence of LPS triggers an innate immune response, activating the immune system and producing cytokines (hormonal regulators). Inflammation is a common reaction to cytokine production, which can also produce host toxicity. The innate immune response to LPS, however, is not synonymous with pathogenicity, or the ability to cause disease. In fact, the innate immune response is triggered purely by LPS.

The therapy of sepsis includes intravenous fluids, antibiotics, surgical drainage of infected fluid collections, and appropriate support for organ dysfunction. Sometimes vasopressors are employed.

In severe sepsis, broad spectrum antibiotics are recommended within 1 hour of making the diagnosis. The type of antibiotic employed is adjusted to culture results. Duration of treatment is typically 7-10 days with the type of antibiotic used directed by the results of cultures.

Due to ever increasing antibiotic resistant strains, and due to the fact that even appropriate antibiotics expose the patient to antibiotic induced endotoxin release, thus exposing the patient to increasing amounts of LPS (endotoxin), an additional strategy for treatment of sepsis has been developed.

LPS

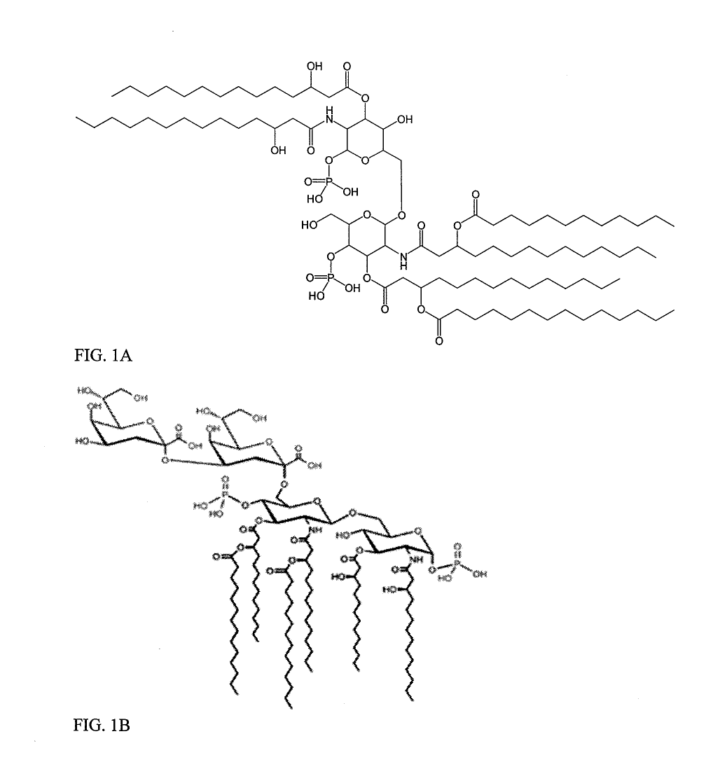

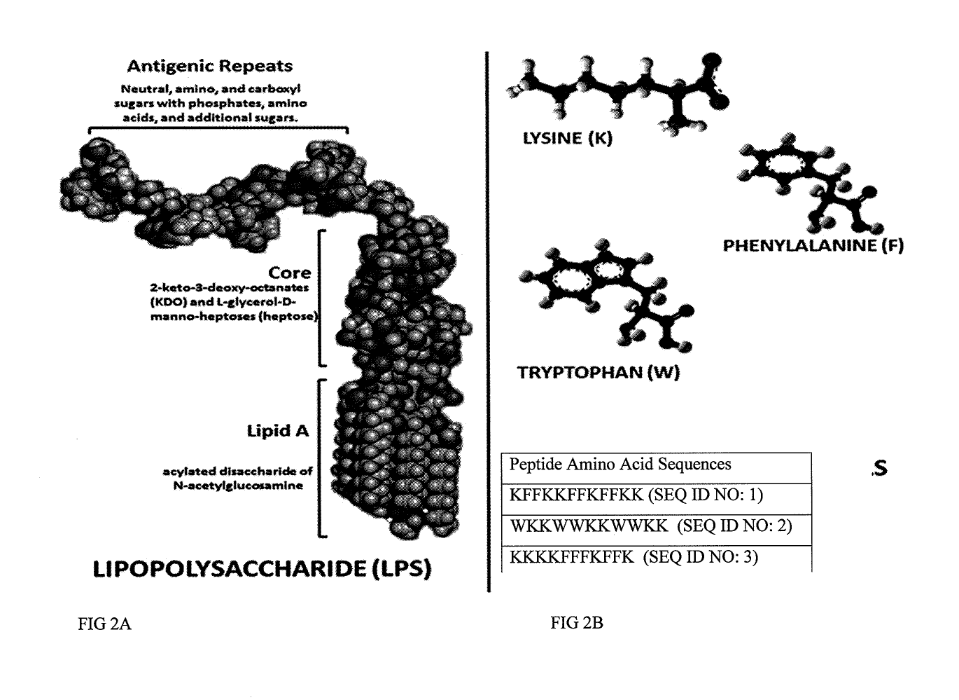

Lipopolysaccharides (LPS), also known as lipoglycans and endotoxin, are large molecules consisting of a lipid portion (Lipid A) and a polysaccharide composed of 0-antigen, outer core and inner core joined by a covalent bond. Endotoxins are responsible for most clinical symptoms of sepsis. LPS is found in the outer membrane of gram-negative bacteria. LPS elicits strong immune responses in animals. The Lipid A portion, for example, the Kdo2-Lipid A shown in FIG. 1, is typically a phosphorylated glucosamine disaccharide, with multiple fatty acid substituents. The fatty acid tails are hydrophobic and anchor the LPS into the bacterial membrane, and the rest of the LPS projects from the cell surface. The lipid A portion of LPS is responsible for much of the toxicity of the gram negative bacteria. LPS is an exogenous pyrogen. When the bacterial cells are lysed by the immune system, fragments of membrane containing lipid A are released into the circulation causing, for example, symptoms such as fever, diarrhea, and possibly endotoxic shock (also called septic shock) which can be fatal. The lipid A portion is very conserved component of LPS. Therefore, the lipid A portion of LPS was selected as a target for the treatment of sepsis.

Peptides

In one embodiment, a method of treating sepsis is provided comprising administering a pharmaceutical composition comprising a peptide of formula I. Peptides according to formula I are provided that are capable of binding to lipid A and blocking multiple pathophysiological processes involved in sepsis.

Peptides were designed to bind to Lipid A. Amino acids were selected which could bind to both the anionic portions and the lipid tails of the lipid A. In order to bind to the carboxylate or phosphate anionic portions of LPS, the amino acid lysine was selected as possessing an amino group which is positively charged at physiological pH. In order to interact with the fatty acid tails- or lipid portion-of the LPS, aromatic amino acid residues were employed. In some embodiments, the aromatic amino acid is selected from phenylalanine or tryptophan. The amino acids are arranged in alternating patterns comprising repeating Lys residues alternating with repeating Phe or Trp residues. The amino acids were arranged in a specific partial alternating fashion in a manner predicted to interact with lipid A. In some embodiments, the peptides are less than 20 amino acids in length to minimize immunogenicity.

The peptide according to the present invention is preferably non-immunogenic. The term “non-immunogenic peptide” as used herein refers to a molecule, in particular to a peptide, which does substantially not provoke an immune response in vivo when administered to a human or an animal being. This molecule property can be determined by methods known in the art. For instance, if the administration of a molecule according to the present invention to an animal (e.g. rabbit, mouse) provokes in an animal a substantial increase of antibodies directed against said molecule, said molecule is considered as an “immunogenic peptide”, if, however, substantially no molecule-specific antibodies can be induced in an animal or human upon administration of said molecule, it is considered as a “non-immunogenic peptide”. It is important that the peptides according to the present invention are non-immunogenic because immunogenic peptides are normally eliminated from the body by the immune system.

In some embodiments, non-immunogenic peptides are provided comprising lysine, phenylalanine, and optionally tryptophan residues in a peptide from 8 to 18 amino acids in length, 10 to 15 amino acids, or 11 to 12 amino acids in length.

In some embodiments, a peptide is provided comprising one or more repeating aromatic amino acid(s) “O” residues, alternating with one or more repeating Lysine residues, wherein each O is selected from Phe, Trp or Tyr.

In some embodiments, a peptide is provided comprising a structure according to Formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2.

In some embodiments, n′=2 or 3; j=1 or 2; j′=2; and j″=1 or 2.

In some embodiments, X═H; O¹, O², O³ and O⁴ are each a Trp residue; Z is OH; m=1; n, n′, j, j′, j″ are each=2; and m′ and m″ both=0 (WK-11; SEQ ID NO: 2).

In some embodiments, X═H; O¹ is absent; O², O³ and O⁴ are each a Phe residue; Z is OH; m=0; n and j″ each=1; and n′, j, j′, m′, and m″ are each=2 (KK-12; SEQ ID NO: 1).

In some embodiments, X═H; O¹ and O⁴ are each absent; O² and O³ are each a Phe residue; Z is OH; m=0; n=4, n′=3; j=1; j′=2; j″=1; m′ and m″=0 (KK-11; SEQ ID NO: 3)

In some embodiments, each of O¹, O², O³, and O⁴ are independently selected from a Phe or a Trp residue.

In some embodiments, each of O¹, O², O³, and O⁴ is a Trp residue.

In some embodiments, each of O¹, O², O³, and O⁴ is a Phe residue.

In some embodiments, one or both of O¹ and O⁴ is absent.

In some aspects, the amino acid residue at the amino terminus of the peptide according to Formula I is selected from a Lys, Phe or Trp residue. In some aspects, the amino acid residue at the amino terminus of the peptide is a Lys residue. In some aspects, the amino acid residue at the amino terminus of the peptide according to Formula I is selected from a Phe or Trp residue. In some aspects, the amino acid residue at the amino terminus of the peptide according to Formula I is a Trp residue.

In some aspects, the amino acid residue at the carboxyl-terminus of the peptide according to Formula I is a Lys residue. In some aspects, the amino acid residue at the carboxyl-terminus of the peptide according to Formula I is an aromatic amino acid residue. In some aspects, the amino acid residue at the carboxyl-terminus of the peptide according to Formula I is an aromatic amino acid residue selected from Phe or Trp.

In some embodiments, the amino terminus is a free amino terminus wherein X is H. In some embodiments, N-terminal modifications are employed selected from acetylated, formylated or guanylated N-termini.

Pharmaceutically acceptable salts of the peptides provided herein may include, for example, an inorganic cation at the C-terminal end of the peptide according to the present invention which may be an alkali metal or alkali earth metal cation, preferably a lithium, sodium, potassium, magnesium or calcium cation. These inorganic cations are regularly used to prepare salts of pharmaceutically active substances. The organic cation may be a quaternary ammonium ion. If the N-terminal end of the peptide according to the present invention comprises a positive charge, said charge may be preferably compensated by an equivalent of an inorganic or organic anion. The organic anion can be, for instance, acetate anion.

In some embodiments, the peptide of Formula I comprises an amino acid sequence selected from SEQ ID NO: 1, 2, 3 or 4.

Amino acid sequences for certain peptide embodiments are shown in Table 1.

TABLE 1
Representative Amino Acid Sequences for Peptides
of Formula 1.

| Peptide | | |
|---|---|---|
| No. | Code | Amino Acid Sequence |
| 1 | KK-12 | KFFKKFFKFFKK (SEQ ID NO: 1) |
| 2 | WK-11 | WKKWWKKWWKK (SEQ ID NO: 2) |
| 3 | KK-11 | KKKKFFFKFFK (SEQ ID NO: 3) |
| 4 | WK-12 | WWKKWWKKWWKK (SEQ ID NO: 4) |

In some embodiments, the peptides were designed to form helical configurations. Without being bound by theory, helical configurations were thought to be able to mimic bacteriophage structure tail tip that provides for the anchoring to bacteria via binding of the LPS component of the bacterial cell wall.

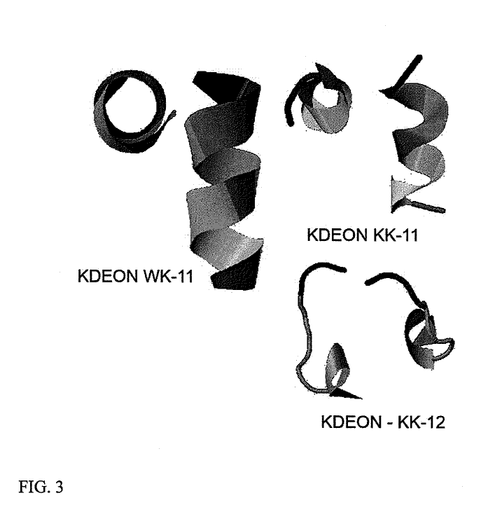

Peptide configurations were subjected to structure modeling representations generated by Mobyle@RPBS, a free bioinformatics software program run by the University of Paris:http://mobyle.rpbs.univ-paris-diderot.fr/cgi-bin/portal.py#forms::PEP-FOLD. Two views of molecular models for each of three peptides, WK-11, KK-11 and KK-12 are shown in FIG. 3. Remarkably, the two sequences (WK-11 and KK-11) which most closely resemble the cylindrical representation (which is a “mimic” of the bacteriophage structure tail tip that provides for the anchoring to bacteria via binding of the LPS component of the bacterial cell wall) also exhibit the two most competitive and specific affinity results from the MS study, where rank order of specific binding to Lipid A was shown to be WK-11≧KK-11>KK-12. The third sequence KK-12, which models with nearly no “cylindericity”, was a very distant third in the competitive binding analysis, as shown in Example 4.

Peptide Synthesis

The peptides of the invention may be prepared by any method known in the art, for example, by solution phase peptide synthesis or by solid phase peptide synthesis. The basic structure of the peptide according to the present invention, which is formed by amino acids, is preferably synthesized chemically according to methods known in the art, e.g. by the method developed by Merrifield et al. (Merrifield, R. B. (1963) J. Am. Chem. Soc. 85, 2149-2154; solid phase peptide synthesis).

The solid phase peptide synthesis method introduced by Merrifield in 1963, for instance, involves the attachment of a growing peptide chain to a solid support. An amino acid corresponding to the C-terminal of the target peptide is covalently attached to an insoluble polymeric support (the “resin”). The next amino acid, with a protected alpha-amino acid, is activated and reacted with the resin-bound amino acid to yield an amino-protected dipeptide on the resin. The amino-protecting group is removed and chain extension is continued with the third and subsequent protected amino acids. After the target protected peptide chain has been built up the resin is cleaved by suitable chemical means thereby releasing the crude peptide product into solution (for solid phase peptide synthesis methods and other peptide synthesis methods see also Fields, G. B. (ed.), Solid Phase Peptide Synthesis in Methods in ENZYMOLOGY, Vol. 289, Academic Press, San Diego (1997); Bodansky, M., Bodansky, A., The practice of peptide synthesis (2nd edn.), Springer Verlag, Berlin (1995); Pennington, M. W., Dunn, B. M. (eds), Peptide Synthesis Protocols, in Methods in Molecular Biology, Vol. 35, Humana Press Inc., Totowa (1994); Grant, G. A. (ed.), Synthetic peptides: a user's guide, W.H. Freemann & Co., New York (1992)).

Amino group protection includes carbamate protection, for example, by tert-butyloxycarbonyl (tBoc or Boc), cleavable, for example, by trifluoroacetic acid (TFA) or anhydrous HF; 9-fluorenylmethyloxycarbonyl (Fmoc) cleavable, for example, by 20% piperidine in DMF; benzyloxy-carbonyl (Z or Cbz) cleavable, for example, by HBr/acetic acid or catalytic hydrogenation; allyloxycarbonyl (Alloc) which is cleavable, for example by tetrakis(triphenylphosphine)palladium (0) in methylene chloride acetic acid and N-methylmorpholine. In some embodiments, the peptide according to formula I, or a pharmaceutically acceptable salt thereof, comprises a Boc, Fmoc, Cbz or Alloc group on the amino-terminus.

Compositions

The peptide according to the present invention may be formulated in a pharmaceutical composition, which can be administered to a patient for preventing or treating sepsis in a patient in need thereof, in particular, a patient suffering from a gram-negative infection.

The pharmaceutical composition may further comprise pharmaceutically acceptable excipients and/or carriers. Suitable excipients and carriers are well known in the art (see e.g. “Handbook of Pharmaceutical Excipients”, 5th Edition by Raymond C. Rowe, Paul J. Sheskey, Sian C. Owen (2005), APhA Publications).

In some embodiments, a pharmaceutical composition for the treatment or prevention of sepsis in a subject in need thereof is provided, wherein the composition comprises a peptide according to Formula I, or pharmaceutically acceptable salt thereof, as provided herein; and a pharmaceutically acceptable carrier.

In some embodiments, a pharmaceutical composition for the treatment or prevention of sepsis in a subject in need thereof is provided, wherein the composition comprises a peptide according to Formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue;
O¹, O², O³, and O⁴ are each independently selected from a Phe, Trp, or Tyr residue;
X is selected from H, COH, C(NH)NH₂, C(O)CH₃, Cbz, Fmoc, Alloc or Boc, or a bond;
Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2; and a pharmaceutically acceptable carrier.

As used herein, a “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. Examples of pharmaceutical compositions of the present invention suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form should be sterile and should be fluid to the extent that easy syringeability exists. It should also be stable under the conditions of manufacture and storage and be able to be preserved against the contamination of microorganisms, such as bacteria and fungi. The pharmaceutical composition may also be in the form of salts.

The pharmaceutically acceptable carrier can also be a solvent or dispersion medium containing, for example, water, ethanol, polyol (for example, glycerol, propylene glycol, and liquid polyethylene glycol, and the like), suitable mixtures thereof, and vegetable oils.

The peptides are preferably comprised in the composition in an amount between 0.1 ug/g to 100 mg/g, preferably 1 ug/g to 80 mg/g. In any way, the effective dosages for prevention or treatment of human patients can be optimized for given patients or patient collectives according to the routine methods available for the present field.

### Combinations

The composition of the present invention may further comprise at least one additional pharmaceutically active component. The pharmaceutical preparation according to the present invention may comprise, in addition to the peptide according to the present invention, further active components, which may exhibit similar properties when administered to an individual or which may cause other reactions in the treated patient.

In some embodiments, the additional active component is one or more of an antibiotic, antioxidant, vasopressor, steroid, or recombinant human activated protein C.

In some embodiments, the additional active component is an antibiotic or combination of antibiotics selected from ceftriaxone plus azithromycin, moxifloxacin, piperacillin/tazobactam combination plus tobramycin, azithromycin, ampicillin plus tobramycin, Cipro plus tobramycin, piperacillin plus tobramycin, ampicillin plus metronidazole, vancomycin plus metronidazole, ceftazidime plus metronidazole, vancomycin plus tobramycin, vancomycin plus clindamycin, clindamycin plus tobramycin plus metronidazole, ceftriaxone plus vancomycin, tobramycin plus vancomycin, Cipro plus vancomycin, tobramycin plus vancomycin plus metronidazole, Cipro plus vancomycin plus metronidazole, ceftazidime, ceftazidime plus tobramycin, and vancomycin, or ceftazadime plus metronidazole.

In some embodiments, the additional active component is an antioxidant. According to the present invention, e.g., antioxidants like vitamins may be considered as further active components because antioxidants inhibit oxidation or suppress reactions promoted by oxygen, oxygen free radicals, oxygen reactive species including peroxides. Antioxidants, especially lipid-soluble antioxidants, can be absorbed into the cell membrane to neutralize oxygen radicals and thereby protect the membrane. The antioxidants useful in the present invention are preferably vitamin antioxidants that may be selected from the group consisting of all forms of Vitamin A including retinal and 3,4-didehydroretinal, all forms of carotene such as alpha-carotene, beta-carotene, gamma carotene, delta-carotene, all forms of Vitamin C (D-ascorbic acid, L-ascorbic acid), all forms of tocopherol such as Vitamin E (Alpha-tocopherol, 3,4-dihydro-2,5,7,8-tetramethyl-2-(4,8,12-trimethyltri-decyl)-2H-1-benzop-yran-6-ol), beta-tocopherol, gamma-tocopherol, delta-tocopherol, tocoquinone, tocotrienol and Vitamin E esters which readily undergo hydrolysis to Vitamin E such as Vitamin E acetate and Vitamin E succinate, and pharmaceutically acceptable Vitamin E salts such as Vitamin E phosphate, prodrugs of Vitamin A, carotene, Vitamin C, and Vitamin E, pharmaceutically acceptable salts of Vitamin A, carotene, Vitamin C, and Vitamin E, and the like, and mixtures thereof.

In some embodiments, a steroid drug is also administered intravenously with the peptide of the invention, wherein the steroid is selected from one or more of the group consisting of dexamethasone, hydrocortisone, and fludrocortisone.

In some embodiments, the additional active component is one or more of a vasopressor compound. In some embodiments, the vasopressor is selected from one or more of the group consisting of norepinephrine, dopamine, epinephrine, vasopressin, phenylephrine, and dobutamine.

In some embodiments, the additional active component is recombinant human activated protein C.

Administration

In one embodiment, a method is provided for administration to a patient in need thereof of a composition according to the invention comprising the peptide of formula I, or a pharmaceutically acceptable salt thereof, as provided herein, selected from an intravenous, intramuscular, spinal, epidural, transdermal, intranasal, mucosal, parenteral, oral, enteral or rectal route of administration. Depending on the route of administration the pharmaceutical composition according to the present invention may be formulated, for instance, as tablets, capsules, liquids, infusion and suppositories (see e.g. “Pharmaceutical Formulation Development of Compounds” by Sven Frokjaer (1999), CRC; “Handbook of Pharmaceutical Manufacturing Formulations” by Sarfaraz K. Niazi (2004), CRC). For example, the compositions may be formulated for administration by injection or for administration by inhalation.

The active compounds will generally be formulated for parenteral administration, e.g., formulated for injection via the intravenous, intramuscular, sub-cutaneous, intralesional, topical and/or even intraperitoneal routes. The preparation of an aqueous composition that contains the peptide as an active component or ingredient will be known to those of skill in the art in light of the present disclosure. Typically, such compositions may be prepared as injectables, either as liquid solutions or suspensions; solid forms suitable for using to prepare solutions or suspensions upon the addition of a liquid prior to injection can also be prepared; and the preparations can also be emulsified. Topical application for mucosal surfaces such as the eye and mouth can be prepared in liquid solutions or suspensions.

In addition to forms of parenteral administration, such as intravenous or intramuscular injection, other acceptable forms of administering the pharmaceutical composition include, but not limited to: tablets or other solids for oral administration; liposomal formulations; time release capsules; and any other form currently used, including creams.

In some embodiments, formulations for delivery of the peptides of the invention include polyethylene glycol (PEG) nanoparticles, for example, as prepared from silicon based polymers, polyesters, or amphiphilic polymers, for example, as disclosed in U.S. Pat. Nos. 6,962,963 and 6,521,736, each of which is incorporated herein by reference.

One may also use nasal solutions or sprays, aerosols or inhalants in the present invention. Nasal solutions are usually aqueous solutions designed to be administered to the nasal passages in drops or sprays. In addition, preservatives, similar to those used in ophthalmic preparations, and appropriate drug stabilizers, if required, may be included in the formulation.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. For oral therapeutic administration, the active compounds of the pharmaceutical composition may be incorporated with excipients and used in the form of ingestible tablets, buccal tables, troches, capsules, elixirs, suspensions, syrups, wafers, and the like.

In particular, the pharmaceutical composition according to any aspect of the present invention may be for topical administration and may be suitable for treatment of skin and/or mucous membrane(s). The pharmaceutical composition may also be in the form of eye drop(s) composition or solution.

The purified isolated peptides may be used without further modifications or may be diluted in a pharmaceutically acceptable carrier. As the peptides are stable, the peptides according to any aspect of the present invention, may be administered to humans or animals, included in food preparations, pharmaceutical preparations, medicinal and pharmaceutical products, cosmetic products, hygienic products, cleaning products and cleaning agents, as well as any material to which the peptides could be sprayed on or adhered to wherein the inhibition of microbial growth on such a material is desired.

While the invention encompasses administration of peptides to a subject for therapeutic or prophylactic benefit, it also is envisioned that the peptides will have other uses, such as for use in a diagnostic application, preparative application, or in an analytical application.

In one example, the peptide may be employed in a solid phase capacity as covalently attached to a polymeric resin for the purpose of absorbing LPS via the lipid A portion of the molecule. In some embodiments, the peptide is attached to an affinity resin. In some embodiments, the peptide-affinity resin is employed to reduce the amount of LPS, endotoxin, or lipid A, in a solution. In some embodiments, the solution is a biological solution. In some embodiments, the biological solution is a cell lysate, growth medium, or blood serum. In some embodiments, the resin is a gel matrix, e.g. agarose, or derived from agarose. In some embodiments, the peptide is covalently attached to a solid-phase resin that may be employed in a column form or in a batch form, e.g., for removal of endotoxin from a solution in either an analytical or a preparative format. In one aspect, the peptide may be attached by the carboxy-terminus to the solid phase resin. In another aspect, the peptide may be attached by the amino-terminus to the solid phase resin.

In some embodiments, the effective amount of peptide of the invention to reduce the amount of LPS in a solution is one that reduces the amount of LPS, or endotoxin, in the solution by at least 50%, at least 90%, at least 99%, at least 99.5%, or at least 99.9% compared to the amount of LPS in the original solution. The amount of endotoxin in a solution can be evaluated by an LAL (Limulus amebocyte lysate) test method, for example, LAL gel-clot, turbidometric or chromogenic test, or by any test method known in the art. See Dawson 2005 LAL Update Vol 22_No 3 rev 001.

The present invention also provides a kit comprising: at least one peptide; at least one pharmaceutical composition; each as described above, disposed in at least one suitable container. The kit may further comprise at least one additional agent.

The peptides of the present invention may be used alone. However, they can also be used in adjunct therapy, in combination with another agent and/or antibiotic.

Methods of Treating

In one embodiment, a method of treating sepsis in a patient in need thereof is provided comprising administering a therapeutically effective amount of a pharmaceutical composition comprising a peptide of formula I or pharmaceutically acceptable salt thereof, as provided herein, and a pharmaceutically acceptable carrier. In some embodiments, the peptide of the invention is administered to a patient for preventing or treating sepsis in a patient in need thereof, in particular, a patient suffering from a gram-negative infection.

In some embodiments, a method of treating sepsis in a patient in need thereof is provided comprising administering a therapeutically effective amount of a pharmaceutical composition comprising a peptide of formula I, or pharmaceutically acceptable salt thereof, as provided herein, and a pharmaceutically acceptable carrier, and further comprises co-administering at least one additional active component.

Without being bound by theory, in one aspect, the peptides according to formula I are effective for treating sepsis because they are capable of binding to lipid A and thus blocking multiple pathophysiological processes involved in sepsis.

In another embodiment, a method of inhibiting effects of lipid A in a cell is provided, the method comprising exposing the cell to an effective amount of a peptide according to formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2.

In some embodiments, a method of inhibiting binding of lipid A to a cell is provided, the method comprising exposing the cell to an effective amount of a peptide according to formula I:

X—(O¹)ₘ—(K)ₙ—(O²)_{n′}—(K)ⱼ—(O³)_{j′}—(K)_{j″}—(O⁴)_{m′}—(K)_{m′}—Z Formula (I)

or a pharmaceutically acceptable salt thereof, wherein each K is a Lys residue; O¹, O², O³, and O⁴ are each independently selected from a Phe, Trp, or Tyr residue; X is selected from H, COH, C(NH)NH₂, C(O)CH₃, Cbz, Fmoc, Alloc or Boc, or a bond; Z is selected from OH, OR₁, NH₂, NR₁R₂, or a bond, wherein R1 and R2 are each independently selected from H, C₁₋₆ alkyl, C₃₋₇ cycloalkyl, alkyl C₃₋₇ cycloalkyl, alkylaryl, or alkylheteroaryl; m, m′, and m″ are each independently 0, 1, or 2; n and n′ are each independently 1, 2, 3, or 4; and j, j′, and j″ are each independently 1 or 2.

In some embodiments, the cell is provided in vitro or in vivo. In some embodiments, the cell is a human cell in vivo. In some embodiments, the cell is a human cell in vitro. In some aspects, the cell is a mammalian cell. In some aspects, the mammalian cell is selected from a hepatic cell, monocyte, myeloid cell, dendritic cell, endothelial, or epithelial cell. In some aspects, the mammalian cell is a cell that expresses a CD14 receptor on its surface. The presence of the CD14 receptor on human cells is strongly associated with endotoxin sensitivity. Pugin et al., 1993, Lipopolysaccharide activation of human endothelial cells and epithelial cells is mediated by lipopolysaccharide binding protein and soluble CD14 PNAS 90:2744-2748, which is incorporated herein by reference.

In one aspect, the cell is a dendritic cell. For example, Stromal interacting molecule 1 (STIM1) regulates store-operated Ca2+ entry (SOCE). DebRoy et al. demonstrated that STIM1 expression in endothelial cells is increased during sepsis and thus contributes to hyper-permeability. DebRoy et al 2014, J Biol Chem published online Jul. 11, 2014. Endotoxin-induced STIM1 expression in endothelial cells requires cooperative signaling of transcription factors NF-kB and AP1/c-Fos and mediates lung vascular hyper-permeability.

### EXAMPLES

### Example 1

### Preparation of Peptides

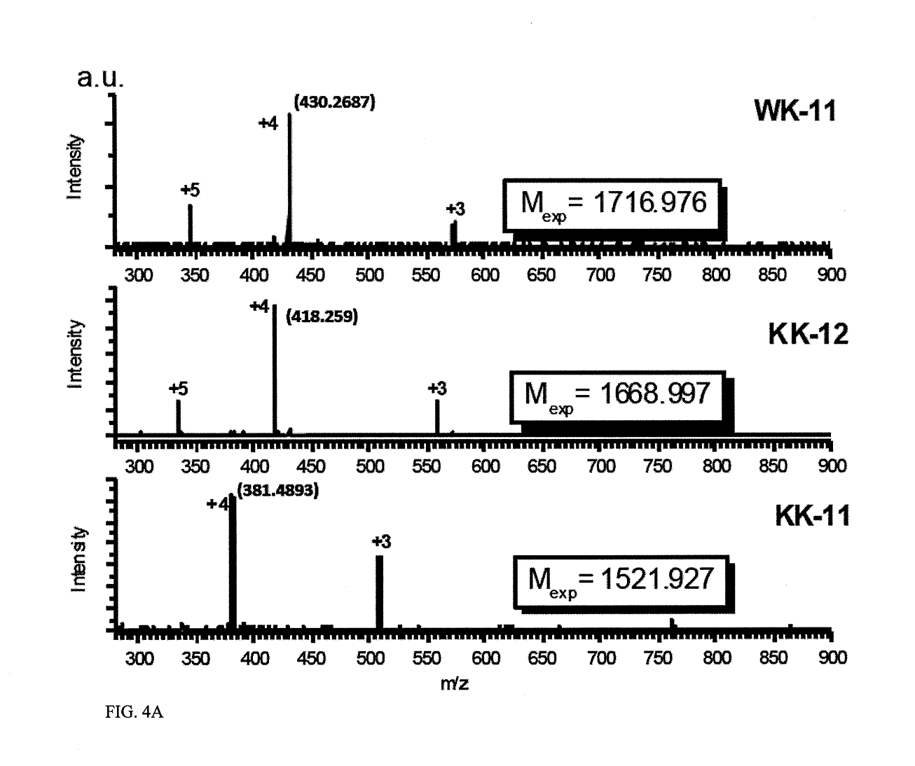

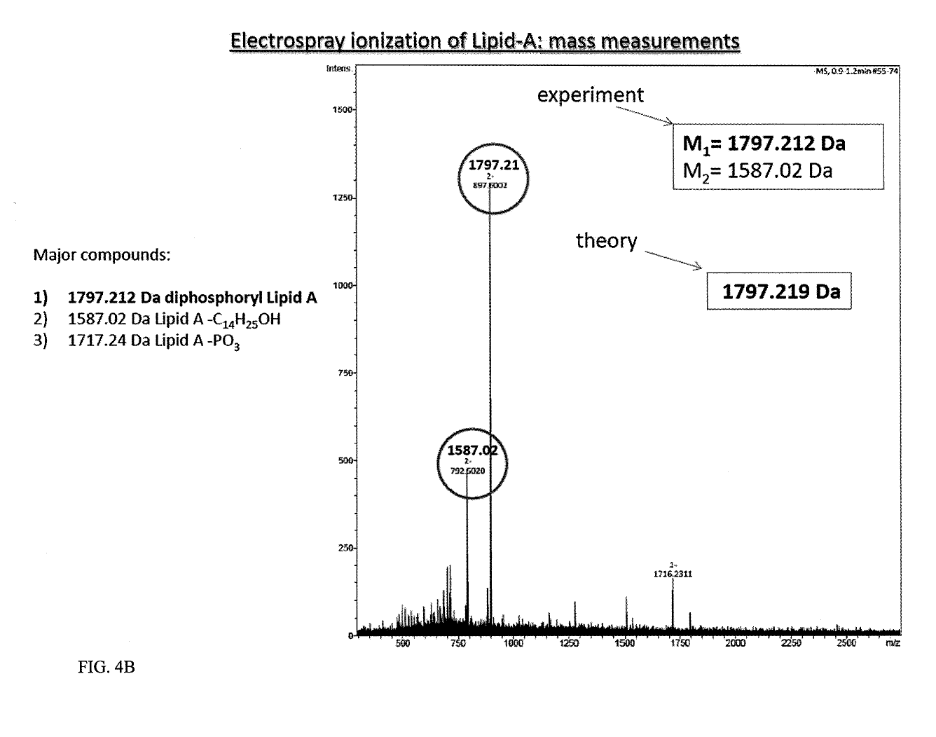

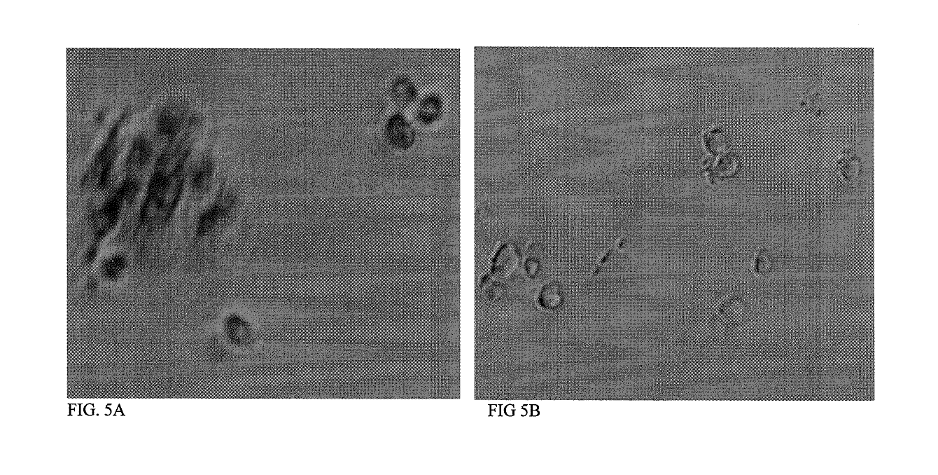

Three peptides a) KFFKKFFKFFKK (SEQ ID NO: 1; KK-12), b) WKKWWKKWWKK (SEQ ID NO: 2; WK-11), and c) KKKKFFFKFFK (SEQ ID NO: 3; KK-11) were prepared by solid phase peptide synthesis and confirmed by electrospray ionization mass spectrometry (ESI-MS). The m/z for each corresponded to the correct structure, as shown in FIG. 4.

### Example 2

### Peptides Block Effect of LPS in THP-1 Cell Line

THP-1 is a human monocytic cell line derived from an acute monocytic leukemia patient. It is typically used to test immunocytochemical analysis of protein-protein interaction and immunohistochemistry. THP-1 cell line can provide continuous culture, grown in suspension, for example, RPMI 1640+10% FBS+2 mM L-glutamine. The average doubling time is 35 to 50 hrs. To inhibit bacterial growth, 1 mM sodium pyruvate, penicillin (100 units/mL) or streptomycin (100 ug/mL) can be employed. Cultures are typically maintained at between 2-9×10⁵ cells/mL at 37 degrees C., and 5% CO₂. The cells are generally non-adhesive.

THP-1 cell line in culture was stimulated with 1 mg/mL LPS. Cells darkened after exposure to LPS, with a rough membrane, and clump or adhere to one another, as shown in FIG. 3A.

In contrast, THP-1 stimulated with LPS (1 mg/mL) and then treated after 24 hours with Peptide 1 (KK-12)(SEQ ID NO: 1) (0.5 mg/mL). After treatment, the cells did not adhere to one another. Cells outer membrane shape and color returned to normal, as shown in FIG. 3B. Therefore, treating cells with Peptide 1 (KK-12)(SEQ ID NO: 1) appears to reverse the effects of LPS.

### Example 3

### Mass Spectrometric (MS) Method to Detect Peptide-Lipid-A Complex Formation

In this example, electrospray ionization mass spectrometry experiments were carried out on Lipid-A (D-Lipid-A) and peptides WK-11, KK-12, KK-11 under a variety of solution and mass spectrometry conditions. Binding of D-Lipid-A to WK-11, KK-12, KK-11 peptides was observed by mass spectrometry methods, as quite abundant ion (LipidA-peptide) complexes were detected from CHL/MeOH/H2O solutions.

Materials: Three peptides KFFKKFFKFFKK (SEQ ID NO: 1; KK-12), WKKWWKKWWKK (SEQ ID NO: 2; WK-11), and KKKKFFFKFFK (SEQ ID NO: 3; KK-11) were ordered from a custom commercial synthesis. Diphosphoryl Lip-A (D-Lipid-A) (C₉₄O₂₅N₂P₂H₁₇₈) was obtained from Sigma. The structure of the D-Lipid-A employed in this example is shown in FIG. 1A.

Chemicals: chloroform (CHL), methanol (MeOH), Water (H2O), triethylamine (TEA), ammonium acetate.

Instrumentation: Micro-TOF ESI-MS (Bruker Daltonics); Q-Star-XL nano-ESI-MS (AB-SCIEX).

Each of each of KK-12, WK-11, and KK-11 was characterized by ESI-MS as shown in FIG. 4A and exhibited characteristic expected m/z peaks. For example, WK-11 (SEQ ID NO: 2) with Mexp=1716.976, exhibited m/z 430.2687 (M+4). KK-12 (SEQ ID NO: 1) with Mexp=1668.997, exhibited m/z 418.259 (M+4). KK-11 (SEQ ID NO: 3) with Mexp=1521.927, exhibited m/z 381.4893 (M+4).

Diphosphoryl Lipid A (C₉₄O₂₅N₂P₂H₁₇₈) was purchased from Sigma and characterized by ESI-MS. Major components in lipid A were diphosphoryl Lipid A (1797.212) Da; Lipid A-C₁₋₄H₂₅OH (1587.02 Da); and Lipid A-PO₃ (1717.24 Da): theoretical 1797.219, as shown in FIG. 4B. Strong signal from corresponding negatively charged ions was measured from CHL/MeOH solution. Diphosphoryl Lipid A was not soluble in water/methanol solution with ammonium acetate (<10 mM), as no signal was registered from its major compound at 1797.21 Da (C₉₄O₂₅N₂P₂H₁₇₈).

Solubility studies were performed in order to optimize the solubility of both the peptides and diphosphoryl Lipid A. Solution conditions for electrospray ionization mass spectrometry experiments on diphosphoryl Lipid A and peptides were evaluated using (CHL)_{X}(MeOH)_{Y}(H2O)_{Z}, where X=0-70%; Y=0-70%; and Z=0-95%.

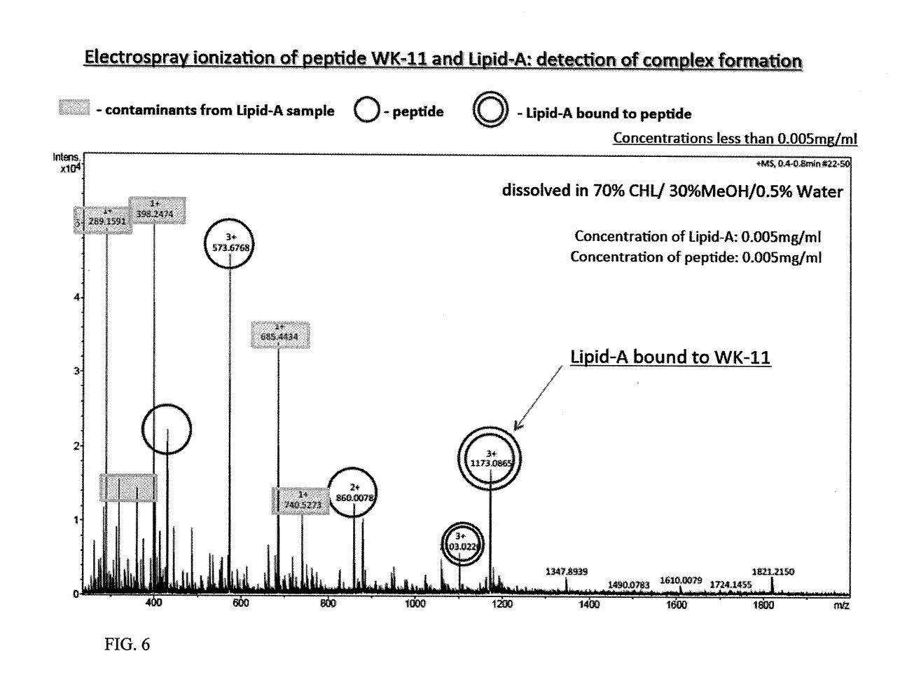

In one experiment, Diphosphoryl Lipid A (0.005 mg/mL) and WK-11 (0.005 mg/mL) were dissolved in 70% CHL/30% MeOH/0.5% water and evaluated by ESI-MS. Electrospray ionization mass spectrometry (ESI-MS) of peptide WK-11 and Lipid-A and detection of complex formation is shown in FIG. 6. Single circles show fragments assigned to the peptide. Double circles show lipid A bound to peptide WK-11. FIG. 6 indicates that peptide WK-11 specifically binds to Lipid A, wherein the WK-11-Lipid-A complex exhibits observed MS (ESI) m/z 1173.0865 (M+3).

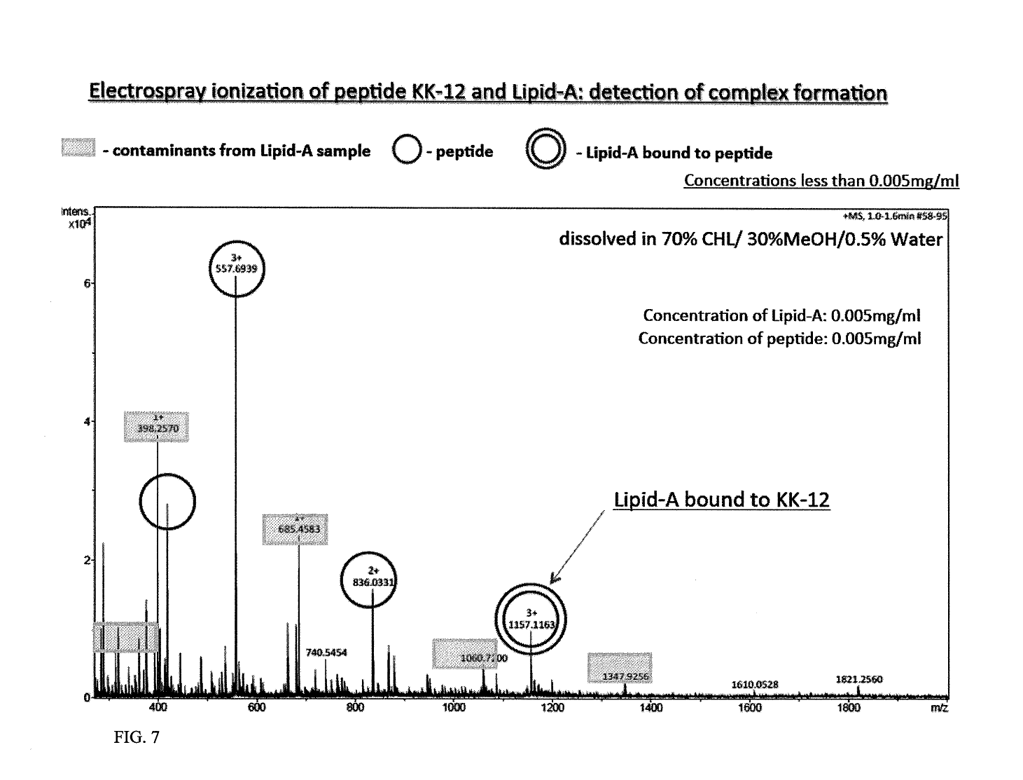

In another experiment, Diphosphoryl Lipid A (0.005 mg/mL) and KK-12 (0.005 mg/mL) were dissolved in 70% CHL/30% MeOH/0.5% water and evaluated by ESI-MS. ESI-MS of peptide KK-12 and Lipid-A and detection of complex formation is shown in FIG. 7. Single circles show fragments assigned to the peptide. Double circles show lipid A bound to peptide KK-12. FIG. 7 indicates that peptide KK-12 specifically binds to Lipid A, wherein the KK-12-Lipid-A complex exhibits observed MS (ESI) m/z 1157.1163 (M+3).

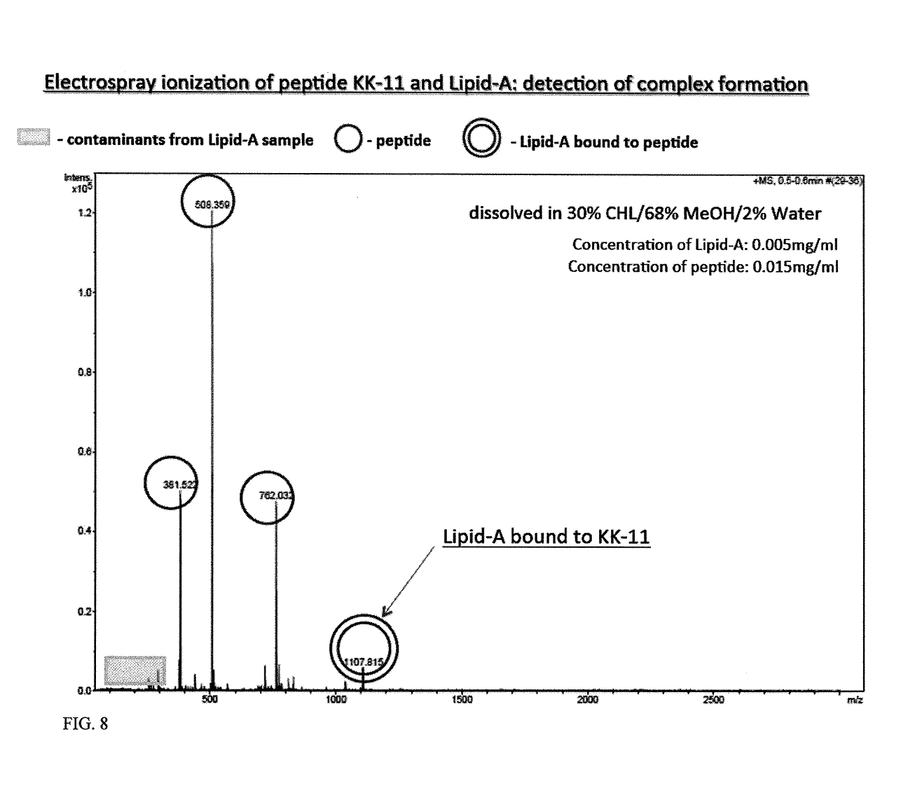

In another experiment, Diphosphoryl Lipid A (0.005 mg/mL) and KK-11 (0.015 mg/mL) were dissolved in 30% CHL/68% MeOH/2% water and evaluated by ESI-MS. Electrospray ionization of peptide KK-11 and Lipid-A and detection of complex formation is shown in FIG. 8. Single circles show fragments assigned to the peptide. Double circles show lipid A bound to peptide KK-11. FIG. 8 indicates that peptide KK-11 specifically binds to Lipid A, wherein the KK-11-Lipid-A complex exhibits observed MS (ESI) m/z 1107.815 (M+3).

Remarkably, binding of diphosphoryl Lipid-A to WK-11, KK-12, and KK-11 peptides was observed by mass spectrometry methods. Lipid-A-peptide complexes exhibited quite abundant ions of the Lipid-A-peptide complex detected in CHL/MeOH/H2O solutions, as shown in FIGS. 6-8.

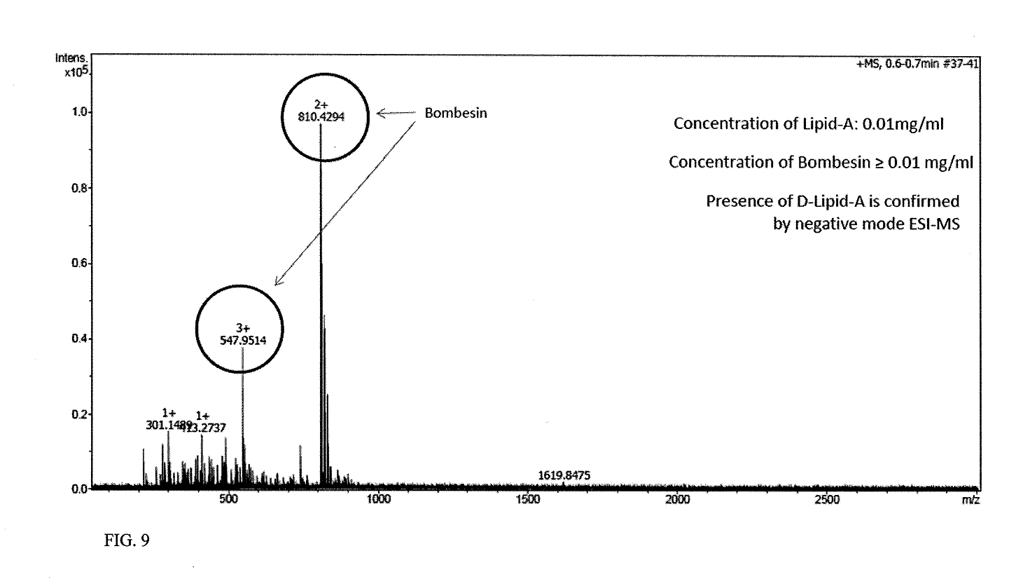

In addition, control standard peptides (Angiotensin, Bombesin, Renin-S with sizes similar to KDEON peptides) showed no sign of binding propensity to D-Lipid-A. A representative ESI-MS ionization for bombesin mixed with D-Lipid-A is shown in FIG. 9.

### Example 4

### Ranking of Lipid A-Binding Propensities of Peptides Using Direct ESI-MS Based Method

ESI-MS based methods were used to rank binding affinities of peptides WK-11, KK-12, and KK-11 to diphosphoryl lipid A in CHL/MeOH/H₂O, 45%/55%/0.05%, unless otherwise specified.

Materials and Chemicals were the same as shown in Example 3.

Instrumentation included Micro-TOF ESI-MS (Bruker Daltonics); FT-ICR-MS NanoDrop-2000 UV-Vis Spectrophotometer.

### Example 4A

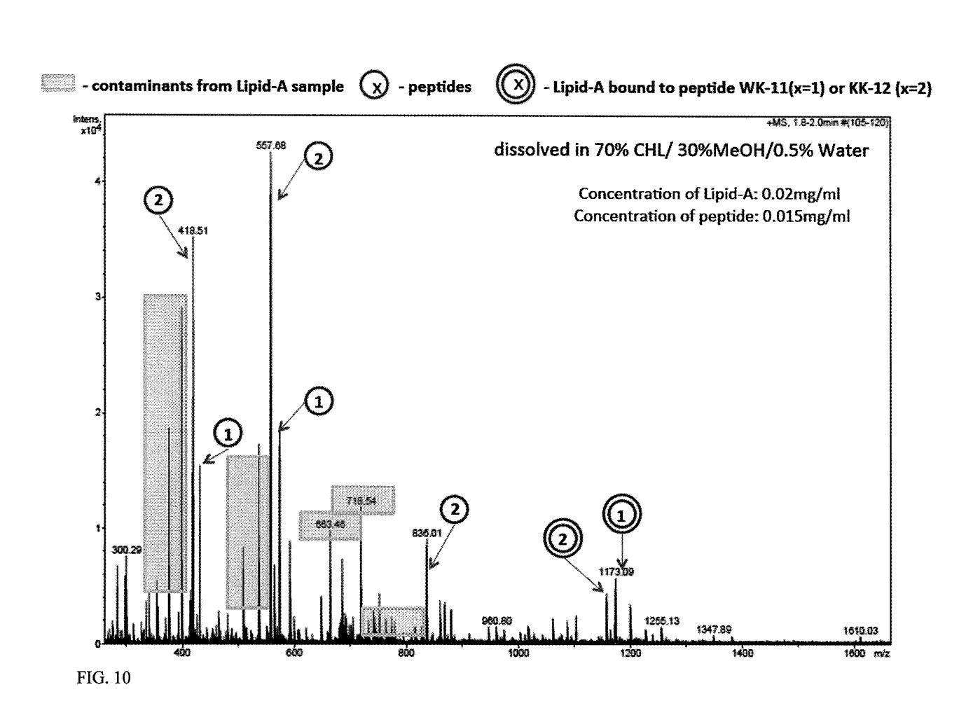

Preliminary Competition experiments for binding to D-lipid-A were conducted using mixtures of peptides via ESI-MS. For example, FIG. 10 shows an ESI-MS competition binding experiment between WK-11 and KK-12 for binding to diphosphoryl Lipid A in 70% CHL/30% MeOH/0.5% water. The Lipid-A was employed at 0.02 mg/mL and the peptides were used at 0.015 mg/mL. Two complexes are observed as shown in FIG. 10, as shown by double circles. The double circle at right surrounding “1” represents a WK-11-Lipid-A complex MS (ESI) m/z 1173.09 (M+3). The double circle surrounding “2” represents KK-12-Lipid-A complex (see comparable MS(ESI) m/z 1157 (M+3) in FIG. 7).

A series of ESI-MS competition experiments were designed to determine relative binding intensities of peptide-lipid A complexes in ESI-MS while varying the concentration of the D-lipid-A.

### Example 4B

### Competition Between WK-11 and KK-12 Peptides for D-Lipid-A

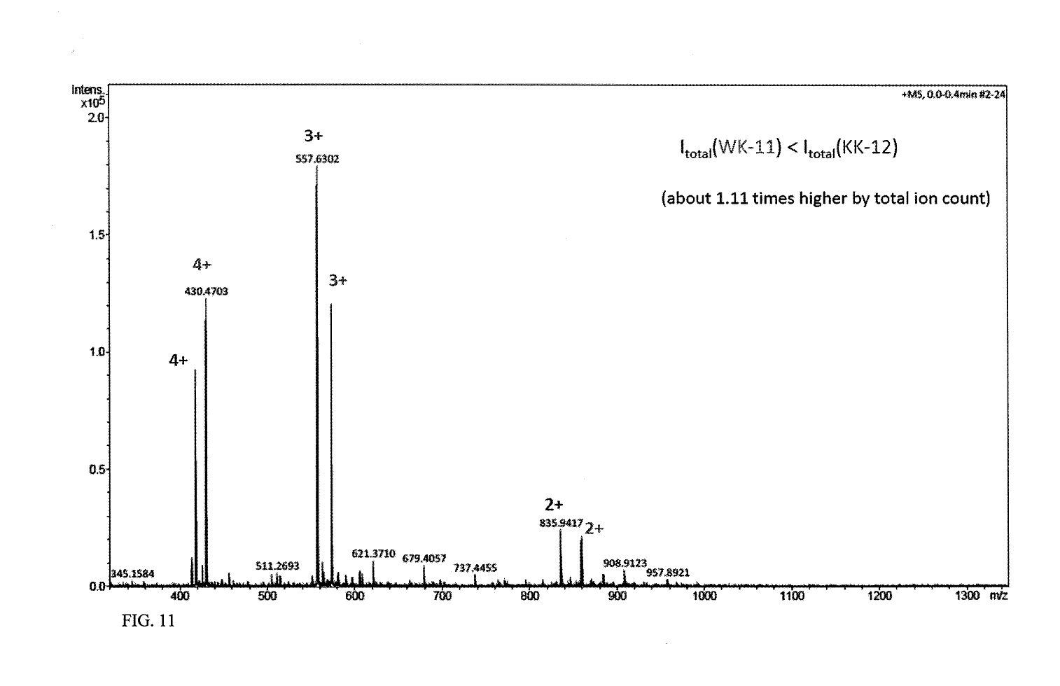

First, signal response intensities from free WK-11 and KK-12 peptides from CHL/MeOH/H2O solutions without D-lipid-A were determined. As shown in FIG. 11, ESI-MS ionization signal response total intensity (Iₜₒₜₐₗ) from free WK-11 and KK-12 peptides from CHL/MeOH/H₂O solutions when no lipid D is present resulted in a difference in signal response intensity was seen between the two peptides where Iₜₒₜₐₗ (WK-11)<Iₜₒₜₐₗ (KK-12) (about 1.11 times higher by total ion count).

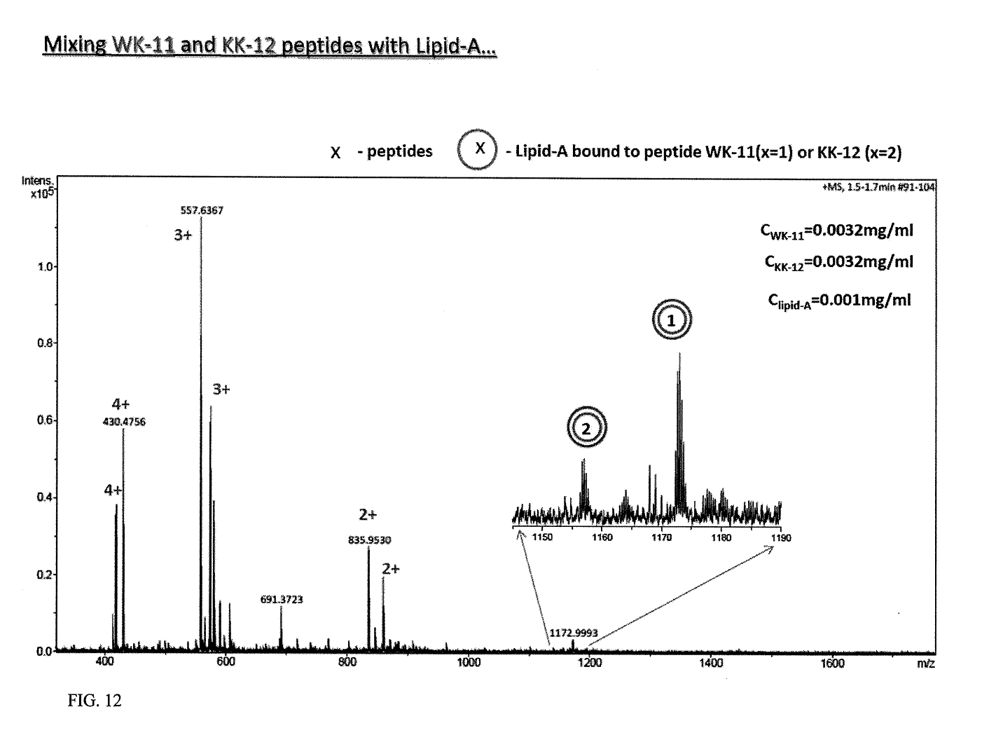

Then, an equal concentration of both peptides was employed, while increasing the concentration of the D-lipid-A in three experiments, as shown in FIGS. 12-14. Double circles surround lipid-A bound to peptide WK-11 (“1”) or lipid-A bound to KK-12 (“2”).

FIG. 12 shows ESI-MS ionization following mixing of WK-11 and KK-12 peptides with diphosphoryl lipid A at 0.0032 mg/mL WK-11; 0.0032 mg/mL KK-12; and 0.001 mg/mL D-lipid-A.

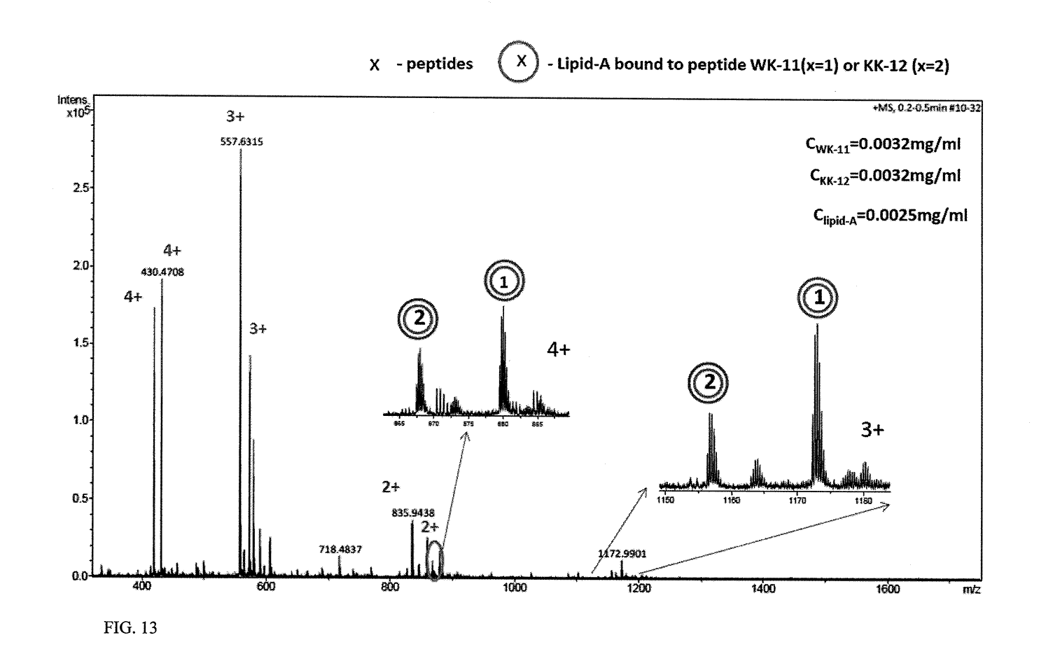

FIG. 13 shows ESI-MS ionization following mixing of WK-11 and KK-12 peptides with diphosphoryl lipid A at 0.0032 mg/mL WK-11; 0.0032 mg/mL KK-12; and 0.0025 mg/mL D-lipid-A.

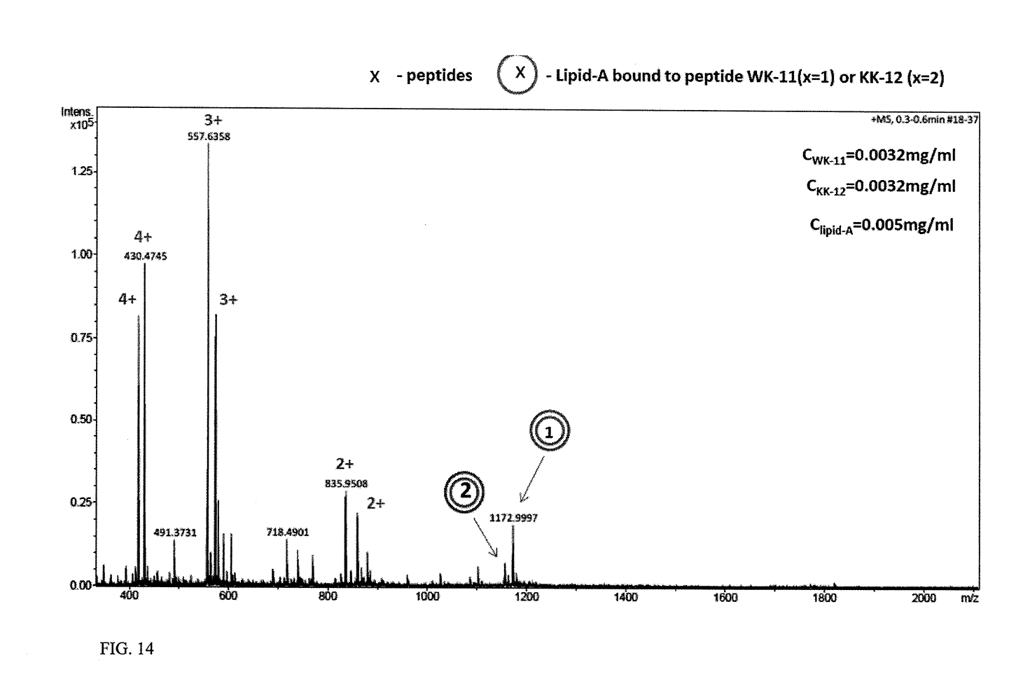

FIG. 14 shows ESI-MS ionization following mixing of WK-11 and KK-12 peptides with diphosphoryl lipid A at 0.0032 mg/mL WK-11; 0.0032 mg/mL KK-12; and 0.0050 mg/mL D-lipid-A.

Resultant signal intensities of peptides and their complexes with D-lipid-A were used to plot R-ratios, determined as in Eq. 1, against D-Lipid-A concentrations.

R═Iₜₒₜₐₗ(peptide:Lipid)/Iₜₒₜₐₗ(peptide) [Eq. 1]

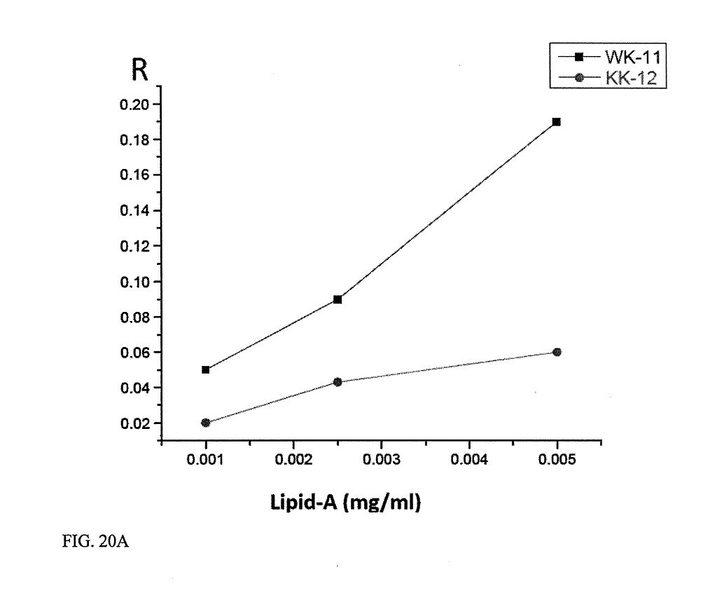

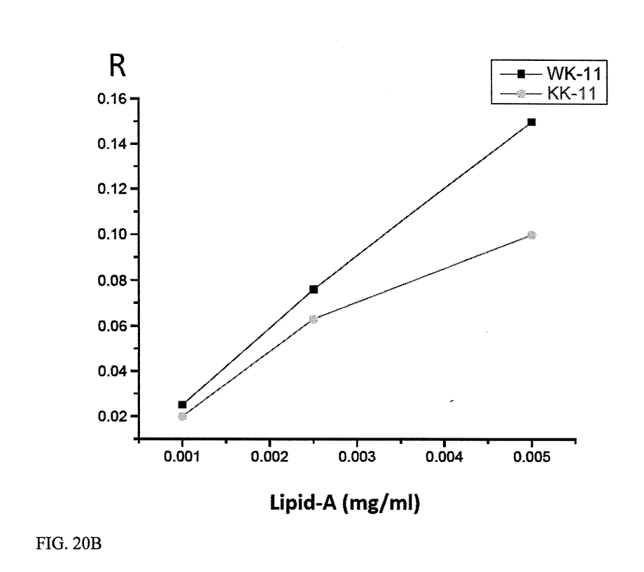

Results for WK-11 and KK-12 are shown in FIG. 20A. Comparison of the corresponding R-functions allows us to rank the peptides Ka binding constants, assuming that the value of the ratio between signal-response factors of complexes is approximately the same or deviates only weakly from the one for corresponding free peptides. It was found that

Ka(WK-11)>Ka(KK-12)

where Ka(x)—relative binding affinity of peptide x with D-Lipid-A.

### Example 4C

### Competition Between WK-11 and KK-11 Peptides for D-Lipid-A

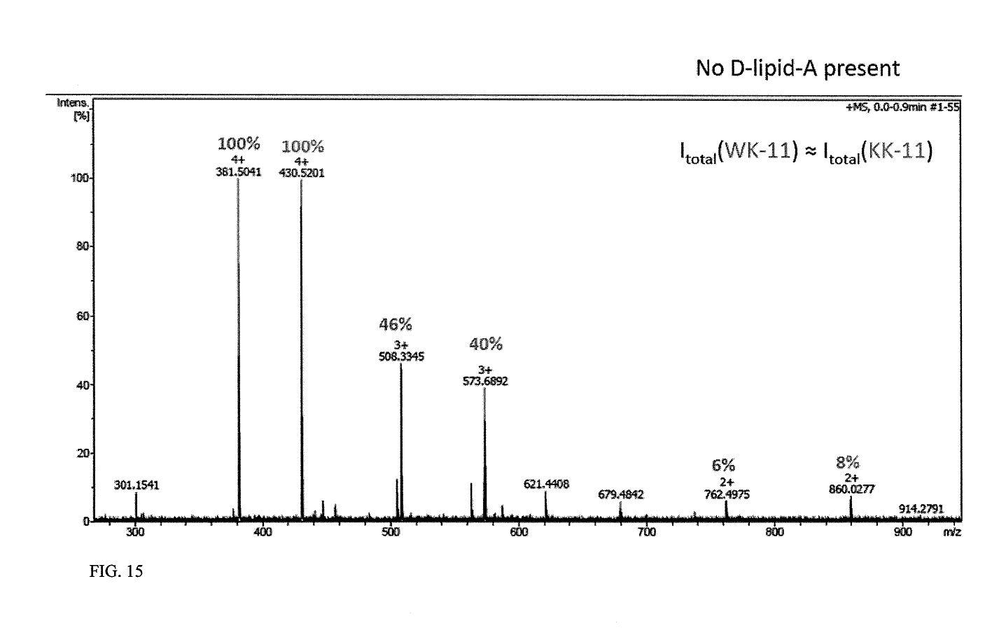

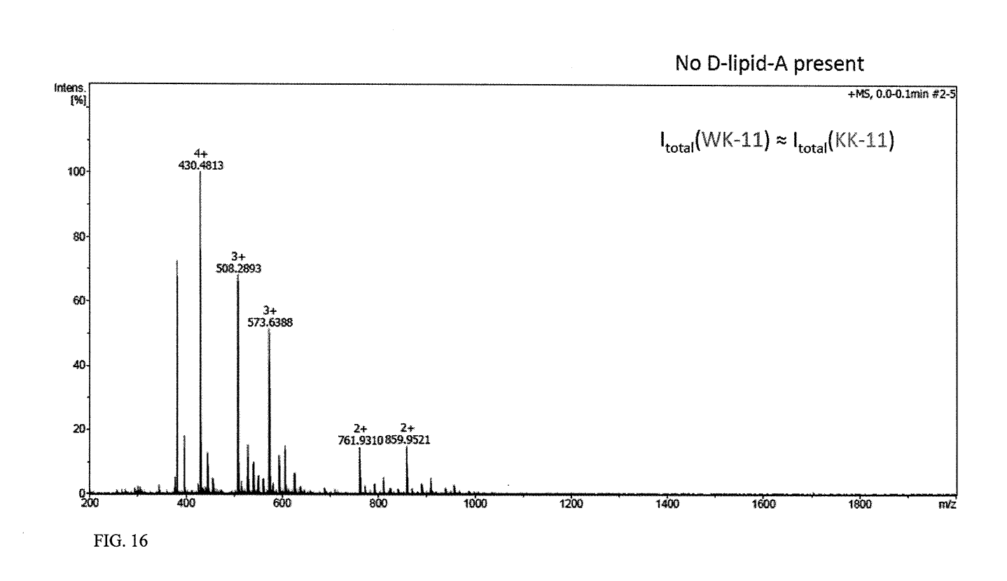

First, signal response from free WK-11 and KK-11 peptides at the same concentration from H2O/MeOH/0.1% formic acid and CHL/MeOH/H2O solutions were determined, as shown in FIGS. 15 and 16.

FIG. 15 shows ESI-MS ionization signal response total intensity (Iₜₒₜₐₗ) from free WK-11 and KK-11 peptides from MeOH/H₂O/0.1% formic acid solution with no D-lipid-A present. Iₜₒₜₐₗ (WK-11)≃Iₜₒₜₐₗ (KK-11).

FIG. 16 shows ESI-MS ionization signal response total intensity (Iₜₒₜₐₗ) from free WK-11 and KK-11 peptides from MeOH/CHL/H₂O solution with no D-lipid-A present. Iₜₒₜₐₗ (WK-11)≃Iₜₒₜₐₗ (KK-11).

Thus, in either solution, peptides WK-11 and KK-11 in solution in the absence of D-lipid-A exhibited relatively equivalent peak ionization signal response intensities.

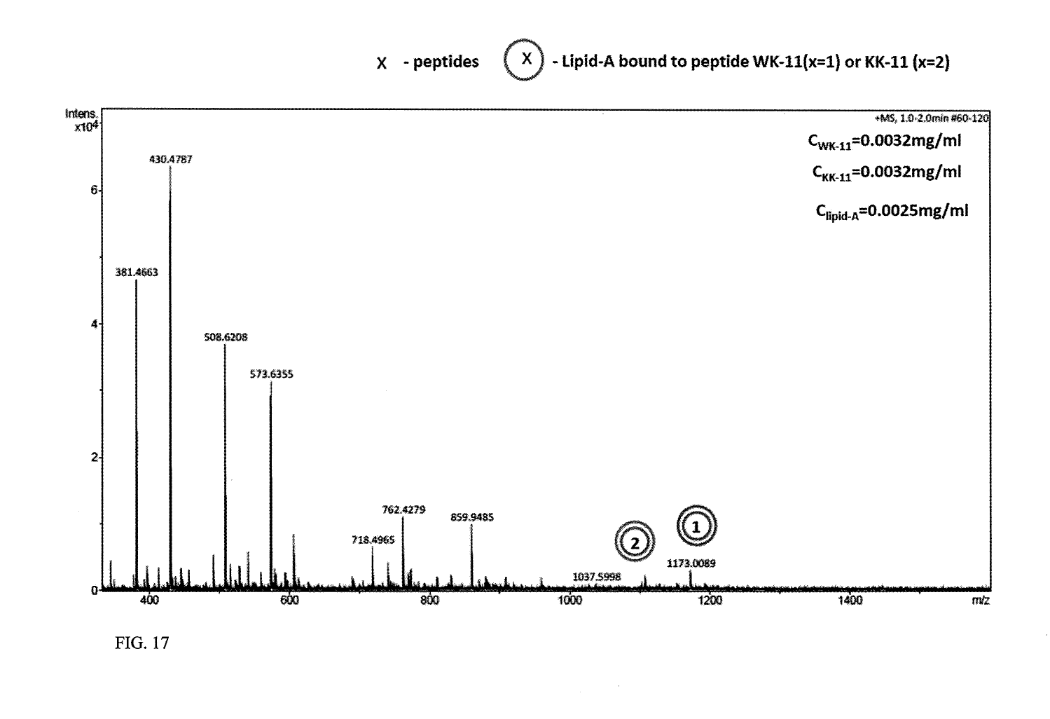

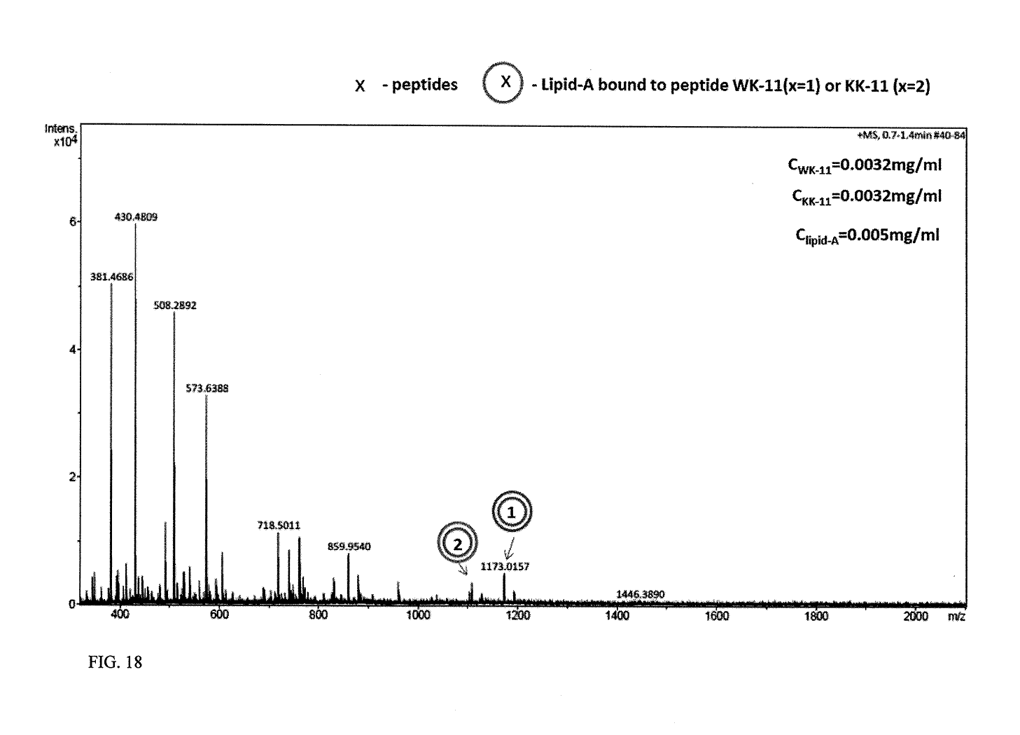

Then, an equal concentration of both peptides was employed, while increasing the concentration of the D-lipid-A in three experiments, two of which are shown in FIGS. 17-18. Double circles surround lipid-A bound to peptide WK-11 (“1”) or lipid-A bound to KK-11 (“2”).

FIG. 17 shows ESI-MS ionization following mixing of WK-11 and KK-11 peptides with diphosphoryl lipid A at 0.0032 mg/mL WK-11; 0.0032 mg/mL KK-11; and 0.0025 mg/mL D-lipid-A.

FIG. 18 shows ESI-MS ionization following mixing of WK-11 and KK-11 peptides with diphosphoryl lipid A at 0.0032 mg/mL WK-11; 0.0032 mg/mL KK-11; and 0.005 mg/mL D-lipid-A.

Resultant signal intensities of peptides and their complexes with D-lipid-A were used to plot R-ratios, determined as in Eq. 1, against D-Lipid-A concentrations, as in Example 4B.

R≃Iₜₒₜₐₗ(peptide:Lipid)/Iₜₒₜₐₗ(peptide) [Eq. 1]

Results for WK-11 and KK-11 are shown in FIG. 20B. Comparison of the corresponding R-functions allows us to rank the peptides Ka binding constants, assuming that the value of the ratio between signal-response factors of complexes is approximately the same or deviates only weakly from the one for corresponding free peptides. It was found that

Ka(WK-11)≧Ka(KK-11)

where Ka(x)—relative binding affinity of peptide x with D-Lipid-A.

### Example 4D

### Competition Between KK-12 and KK-11 Peptides for D-Lipid-A

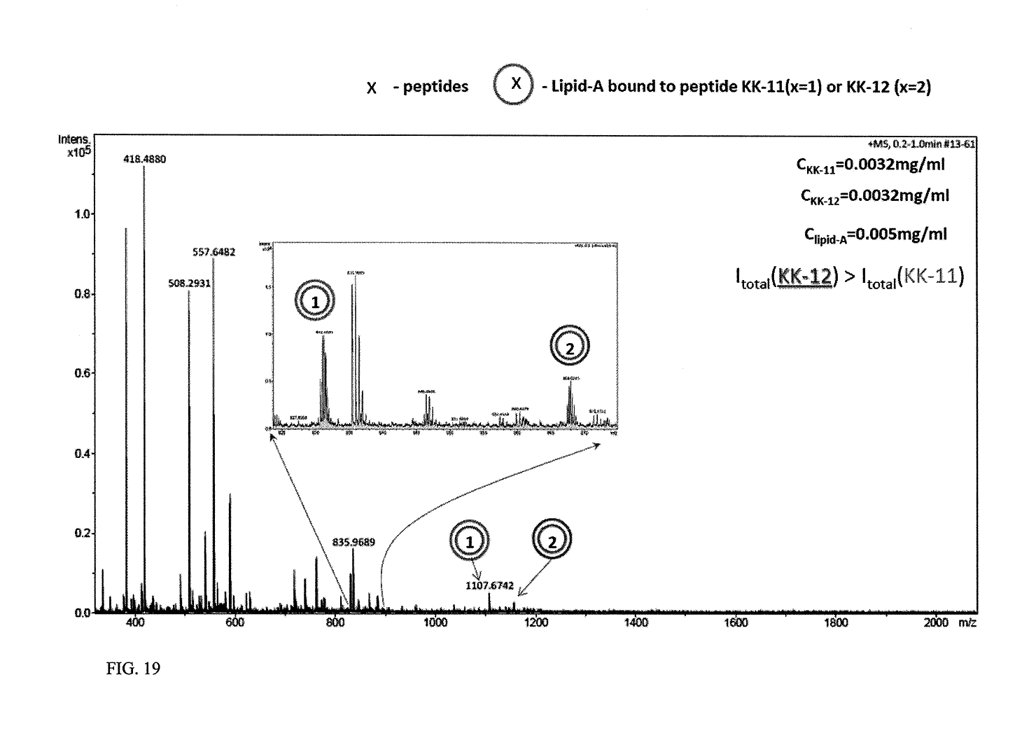

Peptides KK-12 and KK-11 were mixed with D-lipid-A. FIG. 19 shows ESI-MS ionization following mixing of KK-12 and KK-11 peptides with diphosphoryl lipid A at 0.0032 mg/mL KK-12; 0.0032 mg/mL KK-11; and 0.005 mg/mL D-lipid-A. A larger “scattering” of R-values at higher (0.005 mg/ml) concentration of D-lipid-A. ESI-MS competition measurements between KK-11 and KK-12 for D-lipid-A at 0.005 mg/mL corroborate well the finding that Ka(KK-11)>Ka (KK-12).

### Example 4 Conclusion

When taken together, the combined data in Example 4 shows rank order of relative binding affinities (Ka) to D-lipid-A as

Ka(WK-11)≧Ka(KK-11)>Ka(KK-12)

where Ka(x)—relative binding affinity of peptide x with D-Lipid-A.

Remarkably, the two peptides (WK-11 and KK-11) which most closely resemble the cylindrical representation in the modeling studies shown at FIG. 3 (which is a “mimic” of the bacteriophage structure tail tip that provides for the anchoring to bacteria via binding of the LPS component of the bacterial cell wall) also exhibit the two highest binding affinity results from the MS study, where relative binding affinity (Ka) to Lipid A was shown to be Ka (WK-11)≧Ka(KK-11)>Ka(KK-12).

We claim:

1. A peptide of Formula I:
X—(O¹)ₘ—(K)ₙ—(O²)_{n′}—(K)ⱼ—(O³)_{j′}—(K)_{j″}—(O⁴)_{m′}—(K)_{m′}—Z Formula (I)
or a pharmaceutically acceptable salt thereof, wherein
each K is a Lys residue;
O¹, O², O³, and O⁴ are each independently selected from a Phe, Trp, or Tyr residue;
X is selected from H, COH, C(NH)NH₂, C(O)CH₃, Cbz, Fmoc, Alloc or Boc, or a bond;
Z is selected from OH, OR₁, NH₂, NR₁R₂, or a bond, wherein R₁ and R₂ are each independently selected from H, C₁₋₆ alkyl, C₃₋₇ cycloalkyl, alkyl C₃₋₇ cycloalkyl, alkylaryl, or alkylheteroaryl, each optionally substituted with halo;
m, m′, and m″ are each independently 0, 1, or 2;
n and n′ are each independently 1, 2, 3, or 4; and
j, j′, and j″ are each independently 1 or 2.

2. The peptide according to claim 1, or pharmaceutically acceptable salt thereof, wherein each of O¹, O², O³, and O⁴ is independently selected from a Phe or a Trp residue.

3. The peptide according to claim 1, or pharmaceutically acceptable salt thereof, wherein each of O¹, O², O³, and O⁴ is a Trp residue.

4. The peptide according to claim 1, or pharmaceutically acceptable salt thereof, wherein each of O¹, O², O³, and O⁴ is a Phe residue.

5. The peptide according to claim 1, or pharmaceutically acceptable salt thereof, wherein X is H and Z is OH.

6. The peptide according to claim 1, or pharmaceutically acceptable salt thereof, wherein n′=2 or 3; j=1 or 2; j′=2; and j″=1 or 2.

7. The peptide according to claim 1, or pharmaceutically acceptable salt thereof, wherein X═H; O¹, O², and O³ are each a Trp residue; O⁴ is absent; Z is OH; m=1; n, n′, j, j′, j″ are each=2; and m′ and m″ both=0.

8. The peptide according to claim 1, or pharmaceutically acceptable salt thereof, wherein X═H; O¹ is absent; O², O³ and O⁴ are each a Phe residue; Z is OH; m=0; n and j″ each=1; and n′, j, j′, m′, and m″ each=2.

9. The peptide according to claim 1, or pharmaceutically acceptable salt thereof, wherein X═H; O¹ and O⁴ are each absent; O² and O³ are each a Phe residue; Z is OH; m=0; n=4, n′=3; j=1; j′=2; j″=1; m′ and m″=0.

10. The peptide according to claim 1, or pharmaceutically acceptable salt thereof, selected from SEQ ID NO: 1, 2, 3, or 4.

11. A method of inhibiting effects of lipid A on a cell, the method comprising exposing the cell to an effective amount of a peptide according to formula I:
X—(O¹)ₘ—(K)ₙ—(O²)_{n′}—(K)ⱼ—(O³)_{j′}—(K)_{j″}—(O⁴)_{m′}—(K)_{m′}—Z Formula (I)
or a pharmaceutically acceptable salt thereof, wherein
each K is a Lys residue;
O¹, O², O³, and O⁴ are each independently selected from a Phe, Trp, or Tyr residue;
X is selected from H, COH, C(NH)NH₂, C(O)CH₃, Cbz, Fmoc, Alloc or Boc, or a bond;
Z is selected from OH, OR₁, NH₂, NR₁R₂, or a bond, wherein R₁ and R₂ are each independently selected from H, C₁₋₆ alkyl, C₃₋₇ cycloalkyl, alkyl C₃₋₇ cycloalkyl, alkylaryl, or alkylheteroaryl;
m, m′, and m″ are each independently 0, 1, or 2;
n and n′ are each independently 1, 2, 3, or 4; and
j, j′, and j″ are each independently 1 or 2.

12. The method according to claim 11, wherein each of O¹, O², O³, and O⁴ is independently selected from a Phe or a Trp residue.

13. The method according to claim 11, wherein each of O¹, O², O³, and O⁴ is a Trp residue, wherein X is H and Z is OH.

14. The method according to claim 11, wherein n′=2 or 3; j=1 or 2; j′=2; and j″=1 or 2.

15. The method according to claim 11, wherein X═H; O¹, O², and O³ are each a Trp residue; O⁴ is absent; Z is OH; m=1; n, n′, j, j′, j″ are each=2; and m′ and m″ both=0.

16. The method according to claim 11, wherein the peptide is selected from SEQ ID NO: 1, 2, 3, or 4.

17. A pharmaceutical composition comprising a peptide according to Formula I:
X—(O¹)ₘ—(K)ₙ—(O²)_{n′}—(K)ⱼ—(O³)_{j′}—(K)_{j″}—(O⁴)_{m′}—(K)_{m′}—Z Formula (I)
or a pharmaceutically acceptable salt thereof, wherein
each K is a Lys residue;
O¹, O², O³, and O⁴ are each independently selected from a Phe, Trp, or Tyr residue;
X is selected from H, COH, C(NH)NH₂, C(O)CH₃, Cbz, Fmoc, Alloc or Boc, or a bond;
Z is selected from OH, OR₁, NH₂, NR₁R₂, or a bond, wherein R₁ and R₂ are each independently selected from H, C₁₋₆ alkyl, C₃₋₇ cycloalkyl, alkyl C₃₋₇ cycloalkyl, alkylaryl, or alkylheteroaryl;
m, m′, and m″ are each independently 0, 1, or 2;
n and n′ are each independently 1, 2, 3, or 4; and
j, j′, and j″ are each independently 1 or 2; and a pharmaceutically acceptable carrier.

18. The pharmaceutical composition according to claim 17, further comprising an additional active component selected from one or more of an antibiotic, antioxidant, vasopressor, steroid, or recombinant human activated protein C.

19. A method of treating a patient in need thereof, the method comprising Administering to the patient an effective amount of a peptide according to formula I:
X—(O¹)ₘ—(K)ₙ—(O²)_{n′}—(K)ⱼ—(O³)_{j′}—(K)_{j″}—(O⁴)_{m′}—(K)_{m′}—Z Formula (I)
or a pharmaceutically acceptable salt thereof, wherein
each K is a Lys residue;
O¹, O², O³, and O⁴ are each independently selected from a Phe, Trp, or Tyr residue;
X is selected from H, COH, C(NH)NH₂, C(O)CH₃, Cbz, Fmoc, Alloc or Boc, or a bond;
Z is selected from OH, OR₁, NH₂, NR₁R₂, or a bond, wherein R₁ and R₂ are each independently selected from H, C₁₋₆ alkyl, C₃₋₇ cycloalkyl, alkyl C₃₋₇ cycloalkyl, alkylaryl, or alkylheteroaryl;
m, m′, and m″ are each independently 0, 1, or 2;
n and n′ are each independently 1, 2, 3, or 4; and
j, j′, and j″ are each independently 1 or 2.

20. The method according to claim 19, wherein the patient is suffering or suspected of suffering from a gram-negative bacterial infection.
